(19) United States
(12) Patent Application Publication
(10) Pub. No.: US 2025/0224671 A1
(43) Pub. Date: Jul. 10, 2025

(54) ACTINIC RAY-SENSITIVE OR RADIATION-SENSITIVE RESIN COMPOSITION, RESIST FILM, PATTERN FORMING METHOD, AND METHOD FOR PRODUCING ELECTRONIC DEVICE

(71) Applicant: FUJIFILM CORPORATION, Tokyo (JP)

(72) Inventors: Takahiro Mori, Shizuoka (JP); Nishiki Fujimaki, Shizuoka (JP)

(21) Appl. No.: 19/088,008

(22) Filed: Mar. 24, 2025

Related U.S. Application Data
(63) Continuation of application No. PCT/JP2023/037926, filed on Oct. 19, 2023.

(30) Foreign Application Priority Data
Nov. 18, 2022 (JP) 2022-185051

(51) Int. Cl. G03F 7/004; G03F 7/039

(52) U.S. Cl. CPC G03F 7/0045; G03F 7/0046; G03F 7/0397

(57) ABSTRACT

The present invention provides an actinic ray-sensitive or radiation-sensitive resin composition that can form a resist pattern with a low LWR. An actinic ray-sensitive or radiation-sensitive resin composition according to the present invention includes a resin whose polarity is increased by the action of an acid, one or more photoacid generators composed of a cation and an anion and generating an acid upon irradiation with an actinic ray or radiation, a first acid diffusion control agent composed of a first cation and a first anion, and a second acid diffusion control agent composed of a second cation and a second anion, wherein an acidic compound produced by replacing the cation in the photoacid generator with a proton has an acid dissociation constant A of −1.50 or less, an acidic compound produced by replacing the first cation of the first acid diffusion control agent with a proton has an acid dissociation constant B of more than −1.50, and an acidic compound produced by replacing the second cation of the second acid diffusion control agent with a proton has an acid dissociation constant C of more than −1.50, the first cation has a C log P value in the range of 5.400 to 6.000, and the second cation has a C log P value of 8.000 or more.

### CROSS-REFERENCE TO RELATED APPLICATIONS

This application is a Continuation of PCT International Application No. PCT/JP2023/037926 filed on Oct. 19, 2023, which claims priority under 35 U.S.C. § 119(a) to Japanese Patent Application No. 2022-185051 filed on Nov. 18, 2022. The above applications are hereby expressly incorporated by reference, in their entirety, into the present application.

### BACKGROUND OF THE INVENTION

### 1. Field of the Invention

The present invention relates to an actinic ray-sensitive or radiation-sensitive resin composition, a resist film, a pattern forming method, and a method for producing an electronic device.

### 2. Description of the Related Art

After the development of a resist for a KrF excimer laser (248 nm), a pattern forming method utilizing chemical amplification has been used to compensate for a decrease in sensitivity due to light absorption. The pattern forming method is, for example, the following method.

An actinic ray-sensitive or radiation-sensitive resin film (hereinafter also referred to as a “resist film”) formed using an actinic ray-sensitive or radiation-sensitive resin composition (hereinafter also referred to as a “resist composition”) containing a photoacid generator is exposed to light to generate an acid from the photoacid generator. The acid is used as a catalyst to change the solubility of a resin contained in the resist composition in a developer (for example, an alkaline aqueous solution or an organic solvent). The developer is then used to remove an exposed portion or an unexposed portion of the resist film to form a desired pattern.

For example, JP2021-004993A discloses a “resist composition containing a base component whose solubility in a developer is changed by the action of an acid, a compound represented by a specific structure and composed of an anion moiety and a cation moiety, and a fluorine additive containing a fluoropolymer component having a specific constitutional unit”.

### SUMMARY OF THE INVENTION

As a result of studies on the resist composition described in JP2021-004993A, the present inventors have found that there is room for improvement in the line width roughness (LWR) of the resulting resist pattern.

Accordingly, it is an object of the present invention to provide an actinic ray-sensitive or radiation-sensitive resin composition that can form a resist pattern with a low LWR.

It is another object of the present invention to provide a resist film formed using the actinic ray-sensitive or radiation-sensitive resin composition, and a pattern forming method and a device production method using the actinic ray-sensitive or radiation-sensitive resin composition.

As a result of extensive studies to solve the above problems, the present inventors have completed the present invention. That is, the present inventors have found that the above problems can be solved by the following configurations.
[1] An actinic ray-sensitive or radiation-sensitive resin composition containing:
a resin whose polarity is increased by the action of an acid;
one or more photoacid generators;
a first acid diffusion control agent composed of a first cation and a first anion; and
a second acid diffusion control agent composed of a second cation and a second anion,
wherein the photoacid generator is composed of a cation and an anion and generates an acid upon irradiation with an actinic ray or radiation,
an acidic compound produced by replacing the cation in the photoacid generator with a proton has an acid dissociation constant A of −1.50 or less,
an acidic compound produced by replacing the first cation of the first acid diffusion control agent with a proton has an acid dissociation constant B of more than −1.50, and an acidic compound produced by replacing the second cation of the second acid diffusion control agent with a proton has an acid dissociation constant C of more than −1.50,
the first cation has a C log P value in the range of 5.400 to 6.000,
the second cation has a C log P value of 8.000 or more, and
the one or more photoacid generators may be bonded to the first acid diffusion control agent or the second acid diffusion control agent via a covalent bond.
[2] The actinic ray-sensitive or radiation-sensitive resin composition according to [1], wherein a first acid diffusion control agent content is 15% by mole or less based on a total number of moles of the photoacid generator, the first acid diffusion control agent, and the second acid diffusion control agent.
[3] The actinic ray-sensitive or radiation-sensitive resin composition according to [1] or [2], wherein the second cation is a cation containing at least one selected from the group consisting of a fluorine atom and an iodine atom.
[4] The actinic ray-sensitive or radiation-sensitive resin composition according to any one of [1] to [3], wherein the first anion is an anion containing at least one selected from the group consisting of a fluorine atom and an iodine atom.
[5] The actinic ray-sensitive or radiation-sensitive resin composition according to any one of [1] to [4], wherein both the first cation and the second cation are sulfonium cations.
[6]A resist film formed by using the actinic ray-sensitive or radiation-sensitive resin composition according to any one of [1] to [5].
[7]A pattern forming method including:
a step 1 of forming a resist film on a substrate using the actinic ray-sensitive or radiation-sensitive resin composition according to any one of [1] to [5];
a step 2 of exposing the resist film; and
a step 3 of developing the exposed resist film using a developer to form a resist pattern.
[8]A method for producing an electronic device, including the pattern forming method according to [7].

The present invention can provide an actinic ray-sensitive or radiation-sensitive resin composition that can form a resist pattern with a low LWR.

The present invention can also provide a resist film formed using the actinic ray-sensitive or radiation-sensitive resin composition, and a pattern forming method and a method for producing a device using the actinic ray-sensitive or radiation-sensitive resin composition.

### DESCRIPTION OF THE PREFERRED EMBODIMENTS

The present invention is described in detail below.

Although the constituent features described below may be described on the basis of typical embodiments of the present invention, the present invention is not limited to such embodiments.

In the present specification, a numerical range expressed by using “to” means a range including numerical values described before and after “to” as a lower limit and an upper limit.

With respect to the description of a group (atomic group) in the present specification, unless specified as substituted or unsubstituted, the description includes both a group with no substituent and a group with a substituent as long as it is not contrary to the gist of the present invention. For example, the term “alkyl group” includes not only an alkyl group with no substituent (unsubstituted alkyl group) but also an alkyl group with a substituent (substituted alkyl group).

Unless otherwise specified, the substituent is preferably a monovalent substituent. The term “organic group”, as used herein, refers to a group including at least one carbon atom.

In the present specification, a halogen atom is, for example, a fluorine atom, a chlorine atom, a bromine atom, or an iodine atom.

The bonding direction of a divalent linking group in the present specification is not limited unless otherwise specified. For example, when Y in a compound represented by the formula “X—Y—Z” is —COO—, Y may be —CO—O— or —O—CO—. Thus, the compound may be “X—CO—O—Z” or “X—O—CO—Z”.

In the present specification, (meth)acrylate refers to acrylate and methacrylate, and (meth)acryl refers to acryl and methacryl.

The term “actinic ray” or “radiation”, as used herein, refers to, for example, an emission-line spectrum of a mercury lamp, far-ultraviolet light represented by an excimer laser, extreme ultraviolet (EUV), X-rays, or an electron beam (EB).

The term “light”, as used herein, refers to an actinic ray or radiation.

Unless otherwise specified, the term “exposure”, as used herein, includes not only exposure to an emission-line spectrum of a mercury lamp, far-ultraviolet light represented by an excimer laser, extreme ultraviolet (EUV), X-rays, or the like, but also drawing with a particle beam, such as an electron beam or an ion beam.

The term “ppm”, as used herein, refers to “parts-per-million (10⁻⁶)”, “ppb” refers to “parts-per-billion (10⁻⁹)”, and “ppt” refers to “parts-per-trillion (10⁻¹²)”.

In the present specification, 1 angstrom is 1×10⁻¹⁰ m.

In the present specification, the weight-average molecular weight (Mw), the number-average molecular weight (Mn), and the dispersity (also referred to as the “molecular weight distribution”) (Mw/Mn) are defined as polystyrene equivalents by GPC measurement using a gel permeation chromatography (GPC) apparatus (HLC-8120GPC manufactured by Tosoh Corporation) (solvent: tetrahydrofuran, flow rate (sample injection volume): 10 μL, column: TSK gel Multipore HXL-M manufactured by Tosoh Corporation, column temperature: 40° C., flow rate: 1.0 mL/min, detector: differential refractive index detector)).

In the present specification, the acid dissociation constant (pKa) refers to pKa in an aqueous solution and is “pKa in a dimethyl sulfoxide (DMSO) solution” when pKa in the aqueous solution cannot be calculated.

pKa can be calculated, for example, by calculation based on the Hammett substituent constant and a database of known literature values and by a molecular orbital calculation method. A specific method of the molecular orbital calculation method may be a method of calculating H⁺ dissociation free energy in an aqueous solution based on a thermodynamic cycle. A method of calculating the H⁺ dissociation free energy is, for example, but not limited to, calculation by a density functional theory (DFT) or another method reported in literature.

In the present specification, pKa is a value calculated based on the Hammett substituent constant and a database of known literature values using the following software package 1.

Software package 1: Advanced Chemistry Development (ACD/Labs) Software V8.14 for Solaris (1994-2007 ACD/Labs)

When pKa cannot be calculated by the above method, a value determined by a molecular orbital calculation method is used. In a specific method using the molecular orbital calculation method, a value determined by using Gaussian 16 based on DFT is used.

As described later, when one or more photoacid generators and the first acid diffusion control agent or the second acid diffusion control agent form a bound product bonded via a covalent bond, the acid dissociation constant A, the acid dissociation constant B, and the acid dissociation constant C are calculated for the bound product. Thus, when the bound product is formed, the acid dissociation constant A, the acid dissociation constant B, and the acid dissociation constant C are values calculated using the software package 1 for an acidic compound produced by replacing all the cation moieties of the bound product with protons.

More specifically, for example, in a bound product of one photoacid generator and one acid diffusion control agent bonded via a covalent bond, two acid dissociation constants are calculated using the software package 1 for an acidic compound produced by replacing all cations contained in the bound product with protons, and it is considered that the acid dissociation constant corresponds to the acid dissociation constant A when the calculated acid dissociation constant is −1.50 or less, and the acid dissociation constant corresponds to the acid dissociation constant B (or the acid dissociation constant C) when the calculated acid dissociation constant is more than −1.50.

In the present specification, the C log P value is a calculated value of a common logarithm log P of a partition coefficient P between 1-octanol and water. Although a known method and known software can be used to calculate the C log P value, unless otherwise specified, in the present invention, a structure is drawn using ChemDrawProfessional (version 20.1.1.125) manufactured by PerkinElmer, Inc., and a value calculated using the software is used.

A “solid component” means a component that forms a resist film and does not include a solvent. A component, even in a liquid state, that forms a resist film is regarded as a solid component.

In the present specification, when simply described as an “acid diffusion control agent”, it represents a concept including the first acid diffusion control agent and the second acid diffusion control agent.

### [Actinic Ray-Sensitive or Radiation-Sensitive Resin Composition]

An actinic ray-sensitive or radiation-sensitive resin composition (hereinafter also referred to as a “resist composition”) according to the present invention is described in detail below.

A resist composition according to the present invention contains
a resin whose polarity is increased by the action of an acid,
one or more photoacid generators,
a first acid diffusion control agent composed of a first cation and a first anion, and
a second acid diffusion control agent composed of a second cation and a second anion,
wherein the photoacid generator is composed of a cation and an anion and generates an acid upon irradiation with an actinic ray or radiation,
an acidic compound produced by replacing the cation in the photoacid generator with a proton has an acid dissociation constant A of −1.50 or less,
an acidic compound produced by replacing the first cation of the first acid diffusion control agent with a proton has an acid dissociation constant B of more than −1.50, and an acidic compound produced by replacing the second cation of the second acid diffusion control agent with a proton has an acid dissociation constant C of more than −1.50,
the first cation has a C log P value in the range of 5.400 to 6.000,
the second cation has a C log P value of 8.000 or more, and
the one or more photoacid generators may be bonded to the first acid diffusion control agent or the second acid diffusion control agent via a covalent bond.

Although the reason why the resist composition with the above configuration can solve the problems of the present invention is not necessarily clear, the present inventors presume the reason as described below.

However, the presumption does not limit the mechanism by which the effects are obtained. In other words, a case where the effects are obtained by a mechanism other than the following is also within the scope of the present invention.

A resist film produced using a resist composition according to the present invention can be subjected to an exposure treatment and, after the exposure treatment, can be subjected to a development treatment using a developer to form a resist pattern. In the exposure treatment, an acid generated from a photoacid generator can increase the polarity of a specific resin, make an exposed portion more hydrophilic than an unexposed portion, and cause a difference in solubility (dissolution contrast) in the developer between the exposed portion and the unexposed portion, thereby forming a pattern. A larger difference in dissolution contrast is preferred due to higher LWR performance.

In the exposure treatment, light emitted through a mask partially leaks and generates an acid from part of the photoacid generator not only in the exposed portion but also in the unexposed portion. An acid generated also in the unexposed portion is not preferred due to a smaller difference in dissolution contrast but can be quenched by incorporating an acid diffusion control agent.

The first acid diffusion control agent has relatively high hydrophilicity, therefore diffuses easily to the hydrophilic exposed portion, and can quench an acid at the boundary between the exposed portion and the unexposed portion.

On the other hand, the second acid diffusion control agent has relatively high hydrophobicity, therefore remains in the hydrophobic unexposed portion without diffusing, and can quench an acid even if the first acid diffusion control agent diffuses to the exposed portion side and the acid remains in the unexposed portion.

Thus, it is surmised that the first acid diffusion control agent can quench an acid in the boundary, and the second acid diffusion control agent can quench an acid in the unexposed portion, thus forming a pattern with high LWR performance.

### [Specific Resin]

A resist composition according to the present invention contains a resin whose polarity is increased by the action of an acid (hereinafter also referred to as a “specific resin”).

The specific resin preferably has a group that is decomposed by the action of an acid to increase the polarity (hereinafter also referred to as an “acid-decomposable group”) and more preferably includes a repeating unit having an acid-decomposable group.

When the specific resin has an acid-decomposable group, in a pattern forming method according to the present invention, typically, when an alkaline developer is used as a developer, a positive-type pattern is suitably formed, and when an organic-based developer is used as a developer, a negative-type pattern is suitably formed.

The repeating unit having an acid-decomposable group may be a repeating unit having an acid-decomposable group or a repeating unit having an acid-decomposable group with an unsaturated bond.

### <Repeating Unit Having Acid-Decomposable Group>

The specific resin preferably includes a repeating unit having an acid-decomposable group.

The acid-decomposable group refers to a group that is decomposed by the action of an acid and generates a polar group. The acid-decomposable group preferably has a structure having a polar group protected with a group that leaves by the action of an acid (leaving group).

Thus, the specific resin preferably has a repeating unit having a group that is decomposed by the action of an acid and generates a polar group. In a resin having this repeating unit, the polarity is increased by the action of an acid, so that the solubility is increased in an alkaline developer and is decreased in an organic solvent.

The polar group is preferably an alkali-soluble group, for example, an acidic group, such as a carboxy group, a phenolic hydroxy group, a fluorinated alcohol group, a sulfonate group, a phosphate group, a sulfonamide group, a sulfonylimide group, an (alkylsulfonyl)(alkylcarbonyl)methylene group, an (alkylsulfonyl)(alkylcarbonyl)imide group, a bis(alkylcarbonyl)methylene group, a bis(alkylcarbonyl)imide group, a bis(alkylsulfonyl)methylene group, a bis(alkylsulfonyl)imide group, a tris(alkylcarbonyl)methylene group, or a tris(alkylsulfonyl)methylene group, or an alcoholic hydroxy group.

Among these, the polar group is preferably a carboxy group, a phenolic hydroxy group, a fluorinated alcohol group (preferably a hexafluoroisopropanol group), or a sulfonate group, more preferably a carboxy group or a phenolic hydroxy group.

The leaving group that leaves by the action of an acid is, for example, a group represented by the formulae (Y1) to (Y4).

—C(Rx₁)(Rx₂)(Rx₃) Formula (Y1):

—C(═O)OC(Rx₁)(Rx₂)(Rx₃) Formula (Y2):

—C(R₃₆)(R₃)(OR₃₈) Formula (Y3):

—C(Rn)(H)(Ar) Formula (Y4):

In the formula (Y1) and the formula (Y2), Rx₁ to Rx₃ each independently denote an alkyl group (linear or branched), a cycloalkyl group (monocyclic or polycyclic), an alkenyl group (linear or branched), or an aryl group (monocyclic or polycyclic). When all of Rx₁ to Rx₃ are alkyl groups (linear or branched), at least two of Rx₁ to Rx₃ are preferably methyl groups.

Among these, Rx₁ to Rx₃ each independently preferably denote an alkyl group or a cycloalkyl group, more preferably a linear alkyl group.

The alkyl group is preferably an alkyl group with 1 to 5 carbon atoms, for example, a methyl group, an ethyl group, a n-propyl group, an isopropyl group, a n-butyl group, an isobutyl group, or a t-butyl group.

The cycloalkyl group may be a monocyclic cycloalkyl group, such as a cyclopentyl group or a cyclohexyl group, or a polycyclic cycloalkyl group, such as a norbornyl group, a tricyclodecanyl group, a tetracyclodecanyl group, a tetracyclododecanyl group, or an adamantyl group.

The aryl group is preferably an aryl group with 6 to 10 carbon atoms, for example, a phenyl group, a naphthyl group, or an anthryl group.

The alkenyl group is preferably a vinyl group.

Two of Rx₁ to Rx₃ may be bonded together to form a monocyclic ring or a polycyclic ring.

A ring formed by bonding two of Rx₁ to Rx₃ is preferably a cycloalkyl group, more preferably a monocyclic cycloalkyl group, such as a cyclopentyl group or a cyclohexyl group, or a polycyclic cycloalkyl group, such as a norbornyl group, a tricyclodecanyl group, a tetracyclodecanyl group, a tetracyclododecanyl group, or an adamantyl group, more preferably a monocyclic cycloalkyl group with 5 or 6 carbon atoms or a polycyclic cycloalkyl group with 6 to 12 carbon atoms.

In the cycloalkyl group formed by bonding two of Rx₁ to Rx₃, one of the methylene groups constituting the ring may be replaced by a heteroatom, such as an oxygen atom or a sulfur atom, a group containing a heteroatom, such as a carbonyl group, a —SO₂— group, or a —SO₃— group, or a vinylidene group. Furthermore, one or more of the ethylene groups constituting the cycloalkane ring may be replaced by a vinylene group.

In a group represented by the formula (Y1) or the formula (Y2), preferably, Rx₁ denotes a methyl group or an ethyl group, and Rx₂ and Rx₃ are bonded together to form the cycloalkyl group.

When the resist composition is, for example, a resist composition for EUV exposure, the group denoted by Rx₁ to Rx₃ and the ring formed by bonding two of Rx₁ to Rx₃ preferably further have a fluorine atom or an iodine atom as a substituent.

In the formula (Y3), R₃₆ to R₃₈ each independently denote a hydrogen atom or a monovalent organic group. R₃₇ and R₃₈ may be bonded together to form a ring.

The monovalent organic group may be an alkyl group, a cycloalkyl group, an aryl group, an aralkyl group, or an alkenyl group.

Preferred embodiments of the alkyl group, the cycloalkyl group, the aryl group, and the aralkyl group are the same as those of the groups denoted by Rx₁ to Rx₃ described above.

R₃₆ is also preferably a hydrogen atom.

In the alkyl group, the cycloalkyl group, the aryl group, and the aralkyl group, one or more methylene groups may be substituted with a heteroatom, such as an oxygen atom or a sulfur atom, or a group containing a heteroatom, such as a carbonyl group, a —SO₂— group, or a —SO₃— group.

R₃₈ may be bonded to another substituent of the main chain of the repeating unit to form a ring. A group formed by bonding R₃₈ to another substituent of the main chain of the repeating unit is preferably an alkylene group, such as a methylene group.

When the resist composition is, for example, a resist composition for EUV exposure, the group denoted by R₃₆ to R₃₈ and the ring formed by R₃₇ and R₃₈ being bonded together preferably further have a fluorine atom or an iodine atom as a substituent.

In the formula (Y4), Ar denotes an aromatic ring group. Rn denotes an alkyl group, a cycloalkyl group, or an aryl group. Rn and Ar may be bonded together to form a non-aromatic ring. Ar preferably denotes an aryl group.

Preferred embodiments of the alkyl group, the cycloalkyl group, and the aryl group are the same as those of the groups denoted by Rx₁ to Rx₃ described above.

When the resist composition is, for example, a resist composition for EUV exposure, the group denoted by Ar and the group denoted by Rn preferably have a fluorine atom or an iodine atom as a substituent.

From the perspective of high acid decomposability of the repeating unit, when a non-aromatic ring is directly bonded to the polar group (or a residue thereof) in the leaving group that protects the polar group, it is also preferable that a ring atom adjacent to a ring atom directly bonded to the polar group (or the residue thereof) in the non-aromatic ring does not have a halogen atom, such as a fluorine atom, as a substituent.

The group that leaves by the action of an acid may be a 2-cyclopentenyl group with a substituent (such as an alkyl group), such as a 3-methyl-2-cyclopentenyl group, or a cyclohexyl group with a substituent (such as an alkyl group), such as a 1,1,4,4-tetramethylcyclohexyl group.

The repeating unit having an acid-decomposable group is preferably a repeating unit represented by the formula (A).

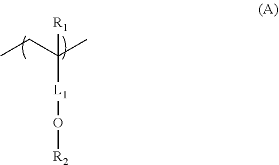

L₁ denotes a divalent linking group, R₁ denotes a hydrogen atom, a fluorine atom, an iodine atom, an alkyl group, or an aryl group, and R₂ denotes a leaving group that leaves by the action of an acid.

L₁ denotes a divalent linking group.

The divalent linking group may be —CO—, —O—, —S—, —SO—, —SO₂—, a divalent hydrocarbon group (for example, an alkylene group, a cycloalkylene group, an alkenylene group, an arylene group, or the like), or a linking group in which a plurality of these groups are linked.

The divalent hydrocarbon group may have a fluorine atom or an iodine atom as a substituent.

Among these, Li preferably denotes —CO—, -Rt-, —COO-Rt-, —COO-Rt-CO—, or -Rt-CO—, more preferably —CO— or —COO-Rt-CO—. Rt denotes a divalent hydrocarbon group, preferably an alkylene group or an arylene group, more preferably an alkylene group.

The arylene group is preferably a phenylene group.

The alkylene group may be linear or branched. The number of carbon atoms in the alkylene group is preferably, but not limited to, in the range of 1 to 10, more preferably 1 to 3.

The total number of fluorine atoms and iodine atoms in the alkylene group having a fluorine atom or an iodine atom is preferably, but not limited to, 2 or more, more preferably 2 to 10, still more preferably 3 to 6.

R₁ denotes a hydrogen atom, a fluorine atom, an iodine atom, an alkyl group, or an aryl group. The alkyl group and the aryl group may have a fluorine atom or an iodine atom as a substituent.

The alkyl group may be linear or branched. The number of carbon atoms in the alkyl group is preferably, but not limited to, in the range of 1 to 10, more preferably 1 to 3.

The total number of fluorine atoms and iodine atoms that the alkyl group may have is preferably, but not limited to, 1 or more, more preferably 1 to 5, still more preferably 1 to 3.

The alkyl group may have a heteroatom, such as an oxygen atom.

R₂ denotes a leaving group that leaves by the action of an acid. The leaving group may have a fluorine atom or an iodine atom as a substituent.

The leaving group may be a leaving group represented by the formulae (Y1) to (Y4).

The repeating unit having an acid-decomposable group is more preferably a repeating unit represented by the formula (AI).

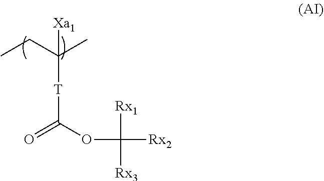

In the formula (AI), Xa₁ denotes a hydrogen atom or an alkyl group optionally having a substituent. T denotes a single bond or a divalent linking group. Rx₁ to Rx₃ each independently denote an alkyl group (linear or branched), a cycloalkyl group (monocyclic or polycyclic), an alkenyl group (linear or branched), or an aryl group (monocyclic or polycyclic).

Two of Rx₁ to Rx₃ may be bonded together to form a monocyclic ring or a polycyclic ring (such as a monocyclic or polycyclic cycloalkyl group).

Xa₁ denotes a hydrogen atom or an alkyl group optionally having a substituent.

The alkyl group optionally having a substituent is, for example, a methyl group or a group represented by —CH₂—R₁₁. R₁₁ denotes a halogen atom, a hydroxy group, or a monovalent organic group. The monovalent organic group is, for example, an alkyl group with 5 or less carbon atoms optionally having a halogen atom, an acyl group with 5 or less carbon atoms optionally having a halogen atom, or an alkoxy group with 5 or less carbon atoms optionally having a halogen atom, preferably an alkyl group with 1 to 3 carbon atoms, more preferably a methyl group.

Xa₁ preferably denotes a hydrogen atom, a methyl group, a trifluoromethyl group, or a hydroxymethyl group.

T denotes a single bond or a divalent linking group.

The divalent linking group may be an alkylene group, an aromatic ring group, —COO-Rt-, or —O-Rt-. Rt denotes an alkylene group or a cycloalkylene group.

T preferably denotes a single bond or —COO-Rt-.

Rt preferably denotes an alkylene group with 1 to 5 carbon atoms, more preferably a methylene group, an ethylene group, or a propylene group.

Rx₁ to Rx₃ each independently denote an alkyl group (linear or branched), a cycloalkyl group (monocyclic or polycyclic), an alkenyl group (linear or branched), or an aryl group (monocyclic or polycyclic).

Two of Rx₁ to Rx₃ may be bonded together to form a monocyclic ring or a polycyclic ring (such as a monocyclic or polycyclic cycloalkyl group).

Preferred embodiments of the alkyl group, the cycloalkyl group, the alkenyl group, and the aryl group denoted by Rx₁ to Rx₃ are the same as those of the groups denoted by Rx₁ to Rx₃ in the formula (Y1) and the formula (Y2).

The cycloalkyl group formed by bonding two of Rx₁ to Rx₃ is preferably a monocyclic cycloalkyl group, such as a cyclopentyl group or a cyclohexyl group, or a polycyclic cycloalkyl group, such as a norbornyl group, a tricyclodecanyl group, a tetracyclododecanyl group, or an adamantyl group. Among these, a monocyclic cycloalkyl group with 5 or 6 carbon atoms or a polycyclic cycloalkyl group with 6 to 12 carbon atoms is preferred.

In the cycloalkyl group formed by bonding two of Rx₁ to Rx₃, for example, one of the methylene groups constituting the ring may be replaced by a heteroatom, such as an oxygen atom or a sulfur atom, a group containing a heteroatom, such as a carbonyl group, a —SO₂— group, or a —SO₃— group, or a vinylidene group. In these cycloalkyl groups, one or more of the ethylene groups constituting the cycloalkane ring may be substituted with a vinylene group.

In the repeating unit represented by the formula (AI), for example, Rx₁ preferably denotes a methyl group or an ethyl group, and Rx₂ and Rx₃ are preferably bonded together to form the cycloalkyl group.

The groups denoted by Rx₁ to Rx₃ may have a substituent. The substituent is, for example, an alkyl group with 1 to 4 carbon atoms, a halogen atom, a hydroxy group, an alkoxy group with 1 to 4 carbon atoms, a carboxy group, or an alkoxycarbonyl group with 2 to 6 carbon atoms.

The repeating unit represented by the formula (AI) is preferably an acid-decomposable (meth)acrylic acid tertiary alkyl ester repeating unit (a repeating unit in which Xa₁ denotes a hydrogen atom or a methyl group and T denotes a single bond or —COO-Rt-).

The repeating unit having an acid-decomposable group is, for example, a repeating unit described in paragraphs [0053] to [0057] of WO2020/158467A.

The specific resin may have a repeating unit having an acid-decomposable group with an unsaturated bond as the repeating unit having an acid-decomposable group.

The repeating unit having an acid-decomposable group with an unsaturated bond is preferably a repeating unit represented by the formula (B).

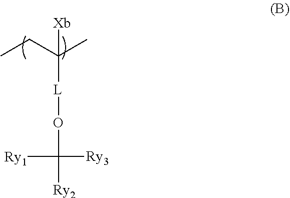

In the formula (B), Xb denotes a hydrogen atom, a halogen atom, or an alkyl group optionally having a substituent. L denotes a single bond or a divalent linking group optionally having a substituent. Ry₁ to Ry₃ each independently denote a linear or branched alkyl group, a monocyclic or polycyclic cycloalkyl group, an alkenyl group, an alkynyl group, or a monocyclic or polycyclic aryl group. At least one of Ry₁ to Ry₃ denotes an alkenyl group, an alkynyl group, a monocyclic or polycyclic cycloalkenyl group, or a monocyclic or polycyclic aryl group.

Two of Ry₁ to Ry₃ may be bonded together to form a monocyclic ring or a polycyclic ring (such as a monocyclic or polycyclic cycloalkyl group or cycloalkenyl group).

Xb denotes a hydrogen atom, a halogen atom, or an alkyl group optionally having a substituent.

A preferred embodiment of Xb is the same as that of Xaᵢ in the formula (AI).

L denotes a single bond or a divalent linking group optionally having a substituent.

The divalent linking group may be an -Rt- group, a —CO— group, a —COO-Rt- group, a —COO-Rt-CO— group, an -Rt-CO— group, or an —O-Rt- group.

Rt denotes an alkylene group, a cycloalkylene group, or an aromatic ring group, preferably an aromatic ring group. Rt may have a substituent, which may be a halogen atom, a hydroxy group, or an alkoxy group.

L preferably denotes an -Rt- group, a —CO— group, a —COO-Rt-CO— group, or an -Rt-CO— group.

Ry₁ to Ry₃ each independently denote an alkyl group (linear or branched), a cycloalkyl group (monocyclic or polycyclic), an alkenyl group, an alkynyl group, a cycloalkenyl group (monocyclic or polycyclic), or an aryl group (monocyclic or polycyclic). At least one of Ry₁ to Ry₃ denotes an alkenyl group, an alkynyl group, a cycloalkenyl group (monocyclic or polycyclic), or an aryl group (monocyclic or polycyclic).

Two of Ry₁ to Ry₃ may be bonded together to form a monocyclic ring or a polycyclic ring (such as a monocyclic or polycyclic cycloalkyl group or cycloalkenyl group).

Preferred embodiments of the alkyl group, the cycloalkyl group, the alkenyl group, and the aryl group denoted by Ry₁ to Ry₃ are the same as those of the groups denoted by Rx₁ to Rx₃ in the formula (Y1).

The alkynyl group denoted by Ry₁ to Ry₃ is preferably an ethynyl group.

The cycloalkenyl group denoted by Ry₁ to Ry₃ preferably has a structure including a double bond in part of a monocyclic cycloalkyl group, such as a cyclopentyl group or a cyclohexyl group.

In the cycloalkyl group or the cycloalkenyl group formed by bonding two of Ry₁ to Ry₃, for example, one of the methylene groups constituting the ring may be replaced by a heteroatom, such as an oxygen atom or a sulfur atom, a group containing a heteroatom, such as a carbonyl group, a —SO₂— group, or a —SO₃— group, a vinylidene group, or a combination thereof. In these cycloalkyl groups or cycloalkenyl groups, one or more of the ethylene groups constituting the cycloalkane ring or the cycloalkene ring may be substituted with a vinylene group.

In the repeating unit represented by the formula (B), for example, Ry₁ preferably denotes a methyl group, an ethyl group, a vinyl group, an allyl group, or an aryl group, and Ry₂ and Ry₃ are preferably bonded together to form the cycloalkyl group or the cycloalkenyl group.

When the groups denoted by Ry₁ to Ry₃ have a substituent, the substituent is preferably an alkyl group with 1 to 4 carbon atoms, a halogen atom, a hydroxy group, an alkoxy group with 1 to 4 carbon atoms, a carboxy group, or an alkoxycarbonyl group with 2 to 6 carbon atoms.

The repeating unit represented by the formula (B) is preferably an acid-decomposable (meth)acrylic acid tertiary ester repeating unit (a repeating unit in which Xb denotes a hydrogen atom or a methyl group and L denotes a —CO— group), an acid-decomposable hydroxystyrene tertiary alkyl ether repeating unit (a repeating unit in which Xb denotes a hydrogen atom or a methyl group and L denotes a phenyl group), or an acid-decomposable styrene carboxylic acid tertiary ester repeating unit (a repeating unit in which Xb denotes a hydrogen atom or a methyl group and L denotes a -Rt-CO— group (Rt denotes an aromatic group)).

When the specific resin includes a repeating unit having an acid-decomposable group with an unsaturated bond, the content thereof preferably ranges from 15% to 80% by mole, more preferably 20% to 70% by mole, still more preferably 30% to 60% by mole, with respect to all the repeating units in the specific resin.

The amount of the repeating unit having an acid-decomposable group is preferably 15% by mole or more, more preferably 20% by mole or more, still more preferably 30% by mole or more, with respect to all the repeating units in the specific resin. The upper limit thereof is preferably 90% by mole or less, more preferably 80% by mole or less, still more preferably 70% by mole or less, particularly preferably 60% by mole or less, with respect to all the repeating units in the specific resin.

The specific resin may include at least one repeating unit selected from the group consisting of the following Group A and/or at least one repeating unit selected from the group consisting of the following Group B.

### Group A: A Group Consisting of the Following Repeating Units (20) to (25).

(20) A repeating unit having an acid group described later
(21) A repeating unit having no acid-decomposable group or acid group and having a fluorine atom, a bromine atom, or an iodine atom described later
(22) A repeating unit having a lactone group, a sultone group, or a carbonate group described later
(23) A repeating unit having a photoacid generating group described later
(24) A repeating unit represented by the formula (V-1) or (V-2) described later
(25) A repeating unit for reducing mobility of a main chain Group B: A group consisting of the following repeating units (30) to (32).
(30) A repeating unit having at least one group selected from the group consisting of a lactone group, a sultone group, a carbonate group, a hydroxy group, a cyano group, and an alkali-soluble group described later
(31) A repeating unit having an alicyclic hydrocarbon structure and having no acid decomposability described later
(32) A repeating unit represented by the formula (III) having no hydroxy group or cyano group described later

The specific resin preferably has an acid group and more preferably includes a repeating unit having an acid group. The specific resin having an acid group has a higher interaction with an acid generated from a photoacid generator. This can further suppress the diffusion of the acid and make the cross-sectional shape of a pattern formed more rectangular.

When a resist composition is used for EUV exposure, the specific resin preferably has at least one repeating unit selected from Group A.

When a resist composition is used for EUV exposure, the specific resin also preferably has a fluorine atom or an iodine atom.

When a resist composition is used for ArF exposure, the specific resin preferably has one repeating unit selected from Group B.

When a resist composition is used for ArF exposure, the specific resin also preferably has no fluorine atom or silicon atom.

When a resist composition is used for ArF exposure, the specific resin also preferably has no aromatic group.

### <Repeating Unit Having Acid Group>

The specific resin may have a repeating unit having an acid group.

The acid group preferably has an acid dissociation constant of 13 or less, more preferably 10 or less, and the lower limit is preferably 3 or more, more preferably 5 or more.

The acid group content of the specific resin is often, but not limited to, in the range of 0.2 to 6.0 mmol/g, preferably 0.8 to 6.0 mmol/g, more preferably 1.2 to 5.0 mmol/g, still more preferably 1.6 to 4.0 mmol/g. An acid group content in the above range results in satisfactory development, a good pattern shape, and high resolution.

The acid group is preferably, for example, a carboxy group, a phenolic hydroxy group, a fluorinated alcohol group (preferably a hexafluoroisopropanol group), a sulfonate group, a sulfonamide group, or an isopropanol group.

In the hexafluoroisopropanol group, one or more (preferably 1 or 2) fluorine atoms may be substituted with a group other than a fluorine atom (such as an alkoxycarbonyl group).

The repeating unit having an acid group preferably has a structure different from the repeating units having an acid-decomposable group and the repeating units having a lactone group, a sultone group, or a carbonate group described later.

The repeating unit having an acid group may have a fluorine atom or an iodine atom.

The repeating unit having an acid group is preferably a repeating unit represented by the following formula (1).

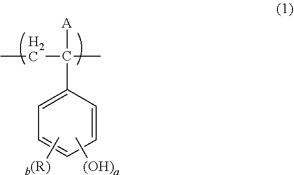

In the formula (1), A denotes a hydrogen atom, an alkyl group, a cycloalkyl group, a halogen atom, or a cyano group.

R denotes a halogen atom, an alkyl group, a cycloalkyl group, an aryl group, an alkenyl group, an aralkyl group, an alkoxy group, an alkylcarbonyloxy group, an alkylsulfonyloxy group, an alkyloxycarbonyl group, or an aryloxycarbonyl group. A plurality of Rs, if present, may be the same or different and may together form a ring. R preferably denotes a hydrogen atom.
a denotes an integer in the range of 1 to 3. b denotes an integer in the range of 0 to (5-a).

The repeating unit having an acid group is, for example, a repeating unit described in paragraphs [0081] to [0086] of WO2020/158467A.

The amount of the repeating unit having an acid group is preferably 10% by mole or more, more preferably 15% by mole or more, with respect to all the repeating units in the specific resin. The upper limit thereof is preferably 70% by mole or less, more preferably 65% by mole or less, still more preferably 60% by mole or less, with respect to all the repeating units in the specific resin.

### <Repeating Unit Having No Acid-Decomposable Group or Acid Group and Having Fluorine Atom, Bromine Atom, or Iodine Atom>

The specific resin may have a repeating unit (hereinafter also referred to as a “unit X”) having no acid-decomposable group or acid group and having a fluorine atom, a bromine atom, or an iodine atom, which is different from the <Repeating Unit Having Acid-Decomposable Group> and the <Repeating Unit Having Acid Group>. The unit X is preferably different from other types of repeating units belonging to Group A, such as <Repeating Unit Having Lactone Group, Sultone Group, or Carbonate Group> and <Repeating Unit Having Photoacid Generating Group> described later.

The unit X is preferably a repeating unit represented by the formula (C).

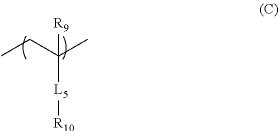

L₅ denotes a single on or —COO—. R₉ denotes a hydrogen atom or an alkyl group optionally having a fluorine atom or an iodine atom. R₁₀ denotes a hydrogen atom, an alkyl group optionally having a fluorine atom or an iodine atom, a cycloalkyl group optionally having a fluorine atom or an iodine atom, an aryl group optionally having a fluorine atom or an iodine atom, or a combined group thereof.

The unit X is, for example, a repeating unit described in paragraph [0093] of WO2020/158467A.

The unit X content is preferably 0% by mole or more, more preferably 5% by mole or more, still more preferably 10% by mole or more, with respect to all the repeating units in the specific resin. The upper limit thereof is preferably 50% by mole or less, more preferably 45% by mole or less, still more preferably 40% by mole or less, with respect to all the repeating units in the specific resin.

### <Repeating Unit Having Lactone Group, Sultone Group, or Carbonate Group>

The specific resin may have a repeating unit having at least one selected from the group consisting of a lactone group, a sultone group, and a carbonate group (hereinafter also referred to as a “unit Y”).

The unit Y also preferably does not have an acid group, such as a hydroxy group or a hexafluoroisopropanol group.

The lactone group or the sultone group may have a lactone structure or a sultone structure. The lactone structure or the sultone structure is preferably a 5- to 7-membered lactone structure or a 5- to 7-membered sultone structure. Among these, a 5- to 7-membered lactone structure fused to another ring structure to form a bicyclo structure or a spiro structure, or a 5- to 7-membered sultone structure fused to another ring structure to form a bicyclo structure or a spiro structure is more preferred.

The specific resin preferably has a repeating unit having a lactone group or a sultone group produced by abstracting one or more hydrogen atoms from a ring atom of a lactone structure represented by any one of the following formulae (LC1-1) to (LC1-21) or a sultone structure represented by any one of the following formulae (SL1-1) to (SL1-3), and the lactone group or the sultone group may be directly bonded to the main chain. For example, a ring atom of the lactone group or the sultone group may constitute the main chain of the specific resin.

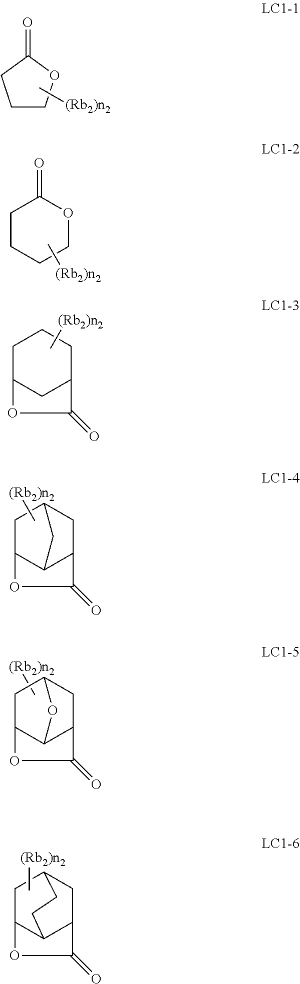

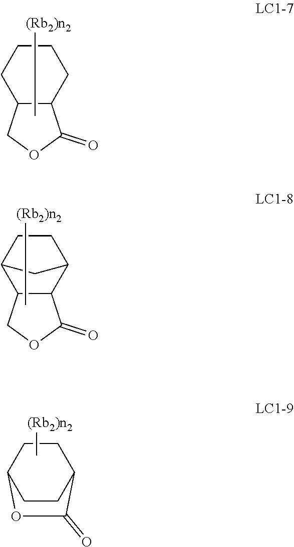

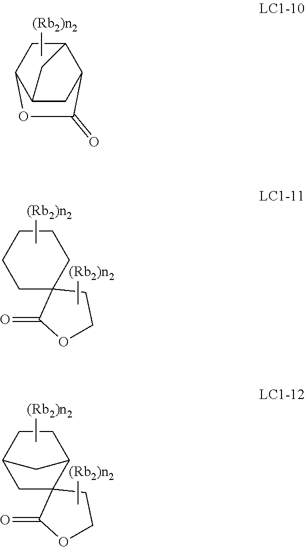

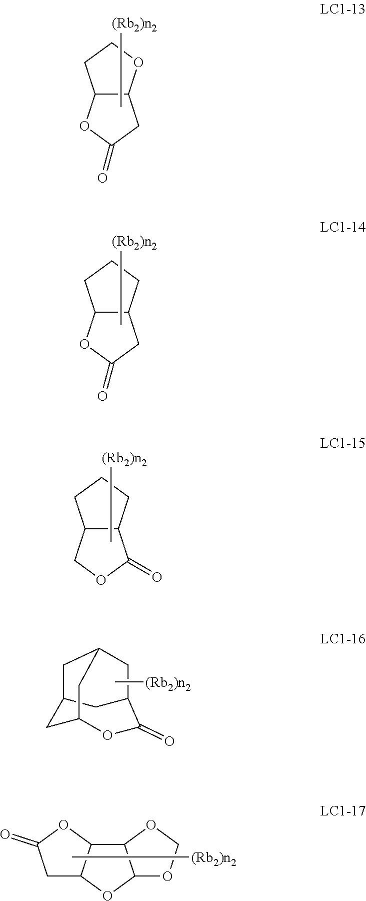

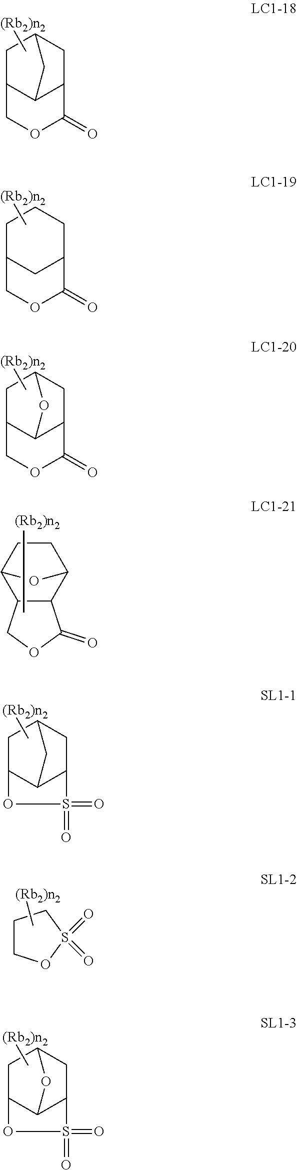

The lactone structure or the sultone structure may have a substituent (Rb₂). A preferred substituent (Rb₂) may be an alkyl group with 1 to 8 carbon atoms, a cycloalkyl group with 4 to 7 carbon atoms, an alkoxy group with 1 to 8 carbon atoms, an alkoxycarbonyl group with 1 to 8 carbon atoms, a carboxy group, a halogen atom, a cyano group, or an acid-decomposable group. n2 denotes an integer in the range of 0 to 4. When n2 is 2 or more, a plurality of Rb₂s may be different from each other or may be bonded together to form a ring.

The repeating unit having a group containing a lactone structure represented by any one of the formulae (LC1-1) to (LC1-21) or a sultone structure represented by any one of the formulae (SL1-1) to (SL1-3) is, for example, a repeating unit represented by the following formula (AI).

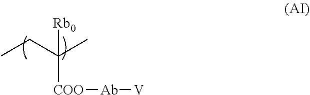

In the formula (AI), Rb₀ denotes a hydrogen atom, a halogen atom, or an alkyl group with 1 to 4 carbon atoms. A preferred optional substituent of the alkyl group in Rb₀ may be a hydroxy group or a halogen atom. The halogen atom may be a fluorine atom, a chlorine atom, a bromine atom, or an iodine atom.

Rb₀ preferably denotes a hydrogen atom or a methyl group.

Ab denotes a single bond, an alkylene group, a divalent linking group having a monocyclic or polycyclic alicyclic hydrocarbon structure, an ether group, an ester group, a carbonyl group, a carboxy group, or a divalent linking group formed by combining these groups. Ab preferably denotes a single bond or a linking group represented by -Ab₁-CO₂—. Ab₁ denotes a linear or branched alkylene group or a monocyclic or polycyclic cycloalkylene group, preferably a methylene group, an ethylene group, a cyclohexylene group, an adamantylene group, or a norbornylene group.

V denotes a group formed by abstracting one hydrogen atom from the ring atoms of a lactone structure represented by any one of the formulae (LC1-1) to (LC1-21) or a group formed by abstracting one hydrogen atom from the ring atoms of a sultone structure represented by any one of the formulae (SL1-1) to (SL1-3).

When an optical isomer is present in the repeating unit having a lactone group or a sultone group, any optical isomer may be used. Furthermore, one optical isomer may be used alone, or a plurality of optical isomers may be mixed and used. When one optical isomer is mainly used, its optical purity (ee) is preferably 90 or more, more preferably 95 or more.

The carbonate group is preferably a cyclic carbonate group.

The unit Y is, for example, a repeating unit described in paragraphs [0104] to [0110] of WO2020/158467A.

The unit Y content is preferably 1% by mole or more, more preferably 10% by mole or more, with respect to all the repeating units in the specific resin. The upper limit thereof is preferably 85% by mole or less, more preferably 80% by mole or less, still more preferably 70% by mole or less, particularly preferably 60% by mole or less, with respect to all the repeating units in the specific resin.

### <Repeating Unit Having Photoacid Generating Group>

The specific resin may have a repeating unit having a group that generates an acid upon irradiation with an actinic ray or radiation (hereinafter also referred to as a “photoacid generating group”) as a repeating unit other than the above repeating units.

The repeating unit having a photoacid generating group may be a repeating unit represented by the formula (4).

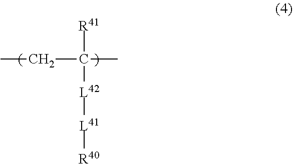

R⁴¹ denotes a hydrogen atom or a methyl group. L⁴¹ denotes a single bond or a divalent linking group. L⁴² denotes a divalent linking group. R⁴⁰ denotes a structural moiety that is decomposed by irradiation with an actinic ray or radiation to generate an acid in a side chain.

Examples of the repeating unit having a photoacid generating group are shown below.

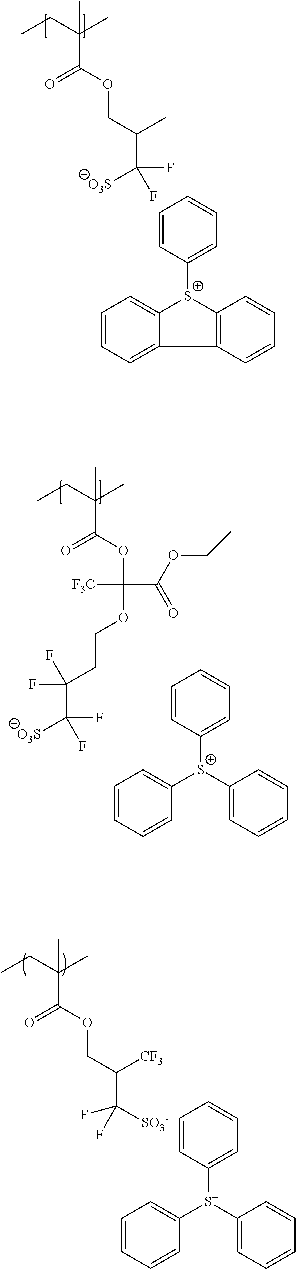

The repeating unit having a photoacid generating group is, for example, a repeating unit described in paragraphs [0094] to [0105] of JP2014-041327A and a repeating unit described in paragraph [0094] of WO2018/193954A.

The amount of the repeating unit having a photoacid generating group is preferably 1% by mole or more, more preferably 5% by mole or more, with respect to all the repeating units in the specific resin. The upper limit thereof is preferably 40% by mole or less, more preferably 35% by mole or less, still more preferably 30% by mole or less, with respect to all the repeating units in the specific resin.

### <Repeating Unit Represented by Formula (V-1) or (V-2)>

The specific resin may have a repeating unit represented by the following formula (V-1) or (V-2).

The repeating units represented by the following formulae (V-1) and (V-2) are preferably repeating units different from the above repeating units.

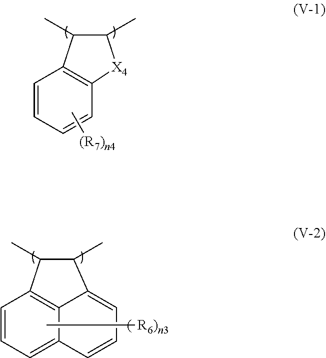

In the formulae, R₆ and R7 each independently denote a hydrogen atom, a hydroxy group, an alkyl group, an alkoxy group, an acyloxy group, a cyano group, a nitro group, an amino group, a halogen atom, an ester group (—OCOR or —COOR: wherein R denotes an alkyl group or a fluorinated alkyl group with 1 to 6 carbon atoms), or a carboxy group. The alkyl group is preferably a linear, branched, or cyclic alkyl group with 1 to 10 carbon atoms.
n3 denotes an integer in the range of 0 to 6.
n4 denotes an integer in the range of 0 to 4.
X4 denotes a methylene group, an oxygen atom, or a sulfur atom.

The repeating unit represented by the formula (V-1) or (V-2) is, for example, a repeating units described in paragraph [0100] of WO2018/193954A.

### <Repeating Unit for Reducing Mobility of Main Chain>

The specific resin preferably has a high glass transition temperature (Tg) from the perspective that excessive diffusion of the generated acid or pattern collapse during development can be suppressed. Tg is preferably more than 90° C., more preferably more than 100° C., still more preferably more than 110° C., particularly preferably 125° C. or more. The upper limit is preferably 400° C. or less, more preferably 350° C. or less, from the perspective of a high dissolution rate in a developer.

In the present specification, Tg of the polymer, such as the specific resin, is calculated by the following method. First, Tg of a homopolymer composed only of each repeating unit contained in the polymer is calculated by the Bicerano method. Next, the mass ratio (%) of each repeating unit to all the repeating units in the polymer is calculated. Next, Tg at each mass ratio is calculated using the Fox equation (described in Materials Letters 62 (2008), 3152 or the like), and the sum total thereof is taken as Tg (° C.) of the polymer.

The Bicerano method is described in Prediction of polymer properties, Marcel Dekker Inc, New York (1993). The calculation of Tg by the Bicerano method can be performed using software MDL Polymer (MDL Information Systems, Inc.) for estimating physical properties of a polymer.

To increase Tg of the specific resin (preferably, Tg higher than 90° C.), the mobility of the main chain of the specific resin is preferably decreased. The method for reducing the mobility of the main chain of the specific resin includes the following methods (a) to (e).
(a) Introduction of a bulky substituent into the main chain
(b) Introduction of a plurality of substituents into the main chain
(c) Introduction of a substituent for inducing interaction between specific resins near the main chain
(d) Formation of a main chain in a ring structure
(e) Linkage of the ring structure to the main chain

The specific resin preferably has a repeating unit in which Tg of a homopolymer is 130° C. or more.

One example of specific means for achieving the above is, for example, a method of introducing a repeating unit described in paragraphs [0107] to [0133] of WO2018/193954A.

<Repeating Unit Having at Least One Group Selected from Lactone Group, Sultone Group, Carbonate Group, Hydroxy Group, Cyano Group, and Alkali-Soluble Group>

The specific resin may have a repeating unit having at least one group selected from a lactone group, a sultone group, a carbonate group, a hydroxy group, a cyano group, and an alkali-soluble group.

The repeating unit having a lactone group, a sultone group, or a carbonate group of the specific resin may be a repeating unit described above in <Repeating Unit Having Lactone Group, Sultone Group, or Carbonate Group>. The preferred content is also as described above in <Repeating Unit Having Lactone Group, Sultone Group, or Carbonate Group>.

The specific resin may have a repeating unit having a hydroxy group or a cyano group from the perspective of further improving the adhesiveness to a substrate and the affinity for a developer.

The repeating unit having a hydroxy group or a cyano group is preferably a repeating unit having an alicyclic hydrocarbon structure substituted with a hydroxy group or a cyano group.

The repeating unit having a hydroxy group or a cyano group preferably does not have an acid-decomposable group. The repeating unit having a hydroxy group or a cyano group is, for example, one described in paragraphs [0081] to [0084] of JP2014-098921A

The specific resin may have a repeating unit having an alkali-soluble group.

The alkali-soluble group may be a carboxy group, a sulfonamide group, a sulfonylimide group, a bissulfonylimide group, or an aliphatic alcohol group substituted with an electron-withdrawing group at the a-position (for example, a hexafluoroisopropanol group), preferably a carboxy group. The specific resin containing a repeating unit having an alkali-soluble group has increased resolution in a contact hole application. The repeating unit having an alkali-soluble group is, for example, one described in paragraphs [0085] and [0086] of JP2014-098921A

### <Repeating Unit Having Alicyclic Hydrocarbon Structure and Having No Acid Decomposability>

The specific resin may have a repeating unit that has an alicyclic hydrocarbon structure and has no acid decomposability. This can reduce the elution of a low-molecular-weight component from a resist film into an immersion liquid during liquid immersion exposure. The repeating unit having an alicyclic hydrocarbon structure and having no acid decomposability is, for example, a repeating unit derived from 1-adamantyl (meth)acrylate, diamantyl (meth)acrylate, tricyclodecanyl (meth)acrylate, or cyclohexyl (meth)acrylate.

### <Repeating Unit Represented by Formula (III) Having No Hydroxy Group or Cyano Group>

The specific resin may have a repeating unit represented by the formula (III) having no hydroxy group or cyano group.

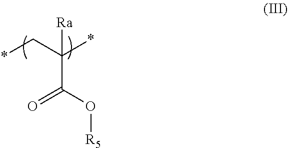

In the formula (III), R5 denotes a hydrocarbon group having at least one ring structure and having no hydroxy group or cyano group.

Ra denotes a hydrogen atom, an alkyl group, or a —CH₂—O—Ra2 group. In the formula, Ra2 denotes a hydrogen atom, an alkyl group, or an acyl group.

The repeating unit represented by the formula (III) having no hydroxy group or cyano group is, for example, a repeating unit described in paragraphs [0087] to [0094] of JP2014-098921A.

### <Other Repeating Units>

The specific resin may have another repeating unit other than the repeating units described above.

For example, the specific resin may have a repeating unit selected from the group consisting of a repeating unit having an oxathiane ring group, a repeating unit having an oxazolone ring group, a repeating unit having a dioxane ring group, and a repeating unit having a hydantoin ring group.

In addition to the above repeating units, the specific resin may have various repeating units for the purpose of adjusting dry etching resistance, suitability for a standard developer, adhesiveness to a substrate, resist profile, resolution, heat resistance, sensitivity, and the like.

The specific resin can be synthesized in the usual manner (for example, radical polymerization).

The weight-average molecular weight of the specific resin as polystyrene equivalent by a GPC method is preferably 30,000 or less, more preferably 1,000 to 30,000, still more preferably 3,000 to 30,000, particularly preferably 5,000 to 15,000.

The specific resin preferably has a dispersity (molecular weight distribution) in the range of 1 to 5, more preferably 1 to 3, still more preferably 1.2 to 3.0, particularly preferably 1.2 to 2.0. As the dispersity decreases, the resolution and the resist shape are improved, and the resist pattern has a smoother sidewall and improved roughness.

The specific resin content preferably ranges from 40.0% to 99.9% by mass, more preferably 60.0% to 90.0% by mass, based on the total solid content of the resist composition.

The specific resin may be used alone or in combination of two or more types thereof.

### [Photoacid Generator]

The resist composition contains at least one photoacid generator composed of a cation and an anion and generating an acid upon irradiation with an actinic ray or radiation.

The photoacid generator is a compound in which the acid dissociation constant A (pKa(A)) of an acidic compound produced by replacing the cation with a proton is −1.50 or less.

The photoacid generator may be in the form of a low-molecular-weight compound or may be in the form of being incorporated into part of a polymer (for example, the specific resin). Furthermore, the form of a low-molecular-weight compound and the form of being incorporated into part of a polymer may be used in combination.

When the photoacid generator is in the form of a low-molecular-weight compound, the photoacid generator preferably has a molecular weight of 3000 or less, more preferably 2000 or less, still more preferably 1,000 or less. The lower limit is preferably, but not limited to, 100 or more.

In the present specification, the photoacid generator is preferably in the form of a low-molecular-weight compound.

The photoacid generator is, for example, a compound (onium salt) represented by “M⁺X⁻”, preferably a compound that generates an organic acid by exposure. The organic acid is, for example, a sulfonic acid (an aliphatic sulfonic acid, an aromatic sulfonic acid, camphorsulfonic acid, or the like).

In the compound represented by “M⁺ X⁻”, M⁺ denotes a cation.

The cation is preferably an organic cation.

In particular, the organic cation is preferably a cation represented by the formula (ZaI) (hereinafter also referred to as a “cation (ZaI)”) or a cation represented by the formula (ZaII) (hereinafter also referred to as a “cation (ZaII)”).

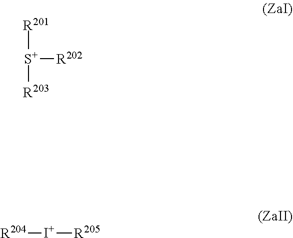

In the formula (ZaI), R²⁰¹, R²⁰², and R²⁰³ each independently denote an organic group. The number of carbon atoms in the organic group preferably ranges from 1 to 30, more preferably 1 to 20. Two of R²⁰¹ to R²⁰³ may be bonded together to form a ring structure, and the ring may include an oxygen atom, a sulfur atom, an ester group, an amide group, or a carbonyl group. The group formed by bonding two of R²⁰¹ to R²⁰³ is, for example, an alkylene group (for example, a butylene group or a pentylene group) or —CH₂—CH₂—O—CH₂—CH₂—. That is, two of R²⁰¹ to R²⁰³ may be bonded together, for example, through a single bond or an ether bond (—O—) to form a ring structure.

A preferred embodiment of the organic cation in the formula (ZaI) may be a cation (ZaI-1), a cation (ZaI-2), a cation (ZaI-3b), or a cation (ZaI-4b) described later.

First, the cation (ZaI-1) is described below.

The cation (ZaI-1) is an arylsulfonium cation in which at least one of R²⁰¹ to R²⁰³ in the formula (ZaI) is an aryl group.

In the arylsulfonium cation, all of R²⁰¹ to R²⁰³ may be an aryl group, or part of R²⁰¹ to R²⁰³ may be an aryl group and the remainder may be an alkyl group or a cycloalkyl group.

One of R²⁰¹ to R²⁰³ may be an aryl group, the remaining two of R²⁰¹ to R²⁰³ may be bonded together to form a ring structure, and the ring may include an oxygen atom, a sulfur atom, an ester group, an amide group, or a carbonyl group. A group formed by bonding two of R²⁰¹ to R²⁰³ is, for example, an alkylene group (for example, a butylene group, a pentylene group, or —CH₂—CH₂—O—CH₂—CH₂—) in which one or more methylene groups may be substituted with an oxygen atom, a sulfur atom, an ester group, an amide group, and/or a carbonyl group.

The arylsulfonium cation may be a triarylsulfonium cation, a diarylalkylsulfonium cation, an aryldialkylsulfonium cation, a diarylcycloalkylsulfonium cation, or an aryldicycloalkylsulfonium cation.

The aryl group in the arylsulfonium cation is preferably a phenyl group or a naphthyl group, more preferably a phenyl group. The aryl group may be an aryl group with a heterocyclic structure having an oxygen atom, a nitrogen atom, a sulfur atom, or the like. The heterocyclic structure may be a pyrrole residue, a furan residue, a thiophene residue, an indole residue, a benzofuran residue, or a benzothiophene residue. When the arylsulfonium cation has two or more aryl groups, the two or more aryl groups may be the same or different.

An alkyl group or a cycloalkyl group that the arylsulfonium cation may have may be a linear alkyl group with 1 to 15 carbon atoms, a branched alkyl group with 3 to 15 carbon atoms, or a cycloalkyl group with 3 to 15 carbon atoms, preferably a methyl group, an ethyl group, a propyl group, a n-butyl group, a sec-butyl group, a t-butyl group, a cyclopropyl group, a cyclobutyl group, or a cyclohexyl group.

A substituent that the aryl group, the alkyl group, or the cycloalkyl group of R²⁰¹ to R²⁰³ may have may be an alkyl group (for example, with 1 to 15 carbon atoms), a cycloalkyl group (for example, with 3 to 15 carbon atoms), an aryl group (for example, with 6 to 14 carbon atoms), an alkoxy group (for example, with 1 to 15 carbon atoms), a cycloalkylalkoxy group (for example, with 1 to 15 carbon atoms), a halogen atom (for example, fluorine or iodine), a hydroxy group, a carboxy group, an ester group, a sulfinyl group, a sulfonyl group, an alkylthio group, or a phenylthio group.

The substituent may further have a substituent, and the alkyl group also preferably has a halogen atom as a substituent to constitute an alkyl halide group, such as a trifluoromethyl group.

The substituent may be an acid-decomposable group. The definition and preferred embodiments of the acid-decomposable group are as described above.

Next, the cation (ZaI-2) is described.

The cation (ZaI-2) is a cation in which R²⁰¹ to R²⁰³ in the formula (ZaI) each independently denote an organic group having no aromatic ring. The aromatic ring also includes an aromatic ring containing a heteroatom.

The number of carbon atoms in the organic group having no aromatic ring preferably ranges from 1 to 30, more preferably 1 to 20.

R²⁰¹ to R²⁰³ preferably each independently denote an alkyl group, a cycloalkyl group, an allyl group, or a vinyl group, more preferably a linear or branched 2-oxoalkyl group, a 2-oxocycloalkyl group, or an alkoxycarbonylmethyl group, still more preferably a linear or branched 2-oxoalkyl group.

The alkyl group or the cycloalkyl group in R²⁰¹ to R²⁰³ is, for example, a linear alkyl group with 1 to 10 carbon atoms or a branched alkyl group with 3 to 10 carbon atoms (for example, a methyl group, an ethyl group, a propyl group, a butyl group, or a pentyl group), or a cycloalkyl group with 3 to 10 carbon atoms (for example, a cyclopentyl group, a cyclohexyl group, or a norbornyl group).

R²⁰¹ to R²⁰³ may be further substituted with a halogen atom, an alkoxy group (for example, with 1 to 5 carbon atoms), a hydroxy group, a cyano group, or a nitro group.

Substituents in R²⁰¹ to R²⁰³ also preferably each independently form an acid-decomposable group by any combination of the substituents.

Next, the cation (ZaI-3b) is described.

The cation (ZaI-3b) is a cation represented by the following formula (ZaI-3b).

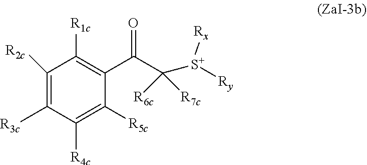

In the formula (ZaI-3b), R_{1c} to R_{5c} each independently denote a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxy group, a nitro group, an alkylthio group, or an arylthio group.

R_{6c} and R_{7c} each independently denote a hydrogen atom, an alkyl group (for example, a t-butyl group), a cycloalkyl group, a halogen atom, a cyano group, or an aryl group.

Rₓ and R_{y} each independently denote an alkyl group, a cycloalkyl group, a 2-oxoalkyl group, a 2-oxocycloalkyl group, an alkoxycarbonylalkyl group, an allyl group, or a vinyl group.

A substituent in R_{1c} to R_{7c}, Rₓ, and R_{y} may be an acid-decomposable group.

Any two or more of R_{1c} to R_{5c}, and Rₓ and R_{y} may be bonded together to form a ring, and the ring may each independently have an oxygen atom, a sulfur atom, an ester group, an amide group, or a carbon-carbon double bond. R_{5c} and R_{6c}, and R_{5c} and Rₓ may be bonded together to form a ring, and the ring also preferably each independently has a carbon-carbon double bond. R_{6c} and R_{7c} may be bonded together to form a ring.

The expression that a ring to be formed has an oxygen atom or the like, for example, means that two groups (for example, Rₓ and R_{y}) capable of being bonded together are bonded together to form an alkylene group, and a methylene group in such an alkylene group is substituted with an oxygen atom or the like.

The ring may be an aromatic or non-aromatic hydrocarbon ring, an aromatic or non-aromatic heterocycle, or a polycyclic fused ring formed by combining two or more of these rings. The ring is preferably a 3- to 10-membered ring, more preferably a 4- to 8-membered ring, still more preferably a 5- or 6-membered ring.

A group formed by any two or more of R_{1c} to R_{5c}, R_{6c} and R_{7c}, and Rₓ and R_{y} bonded together may be a butylene group, a pentylene group, —CH₂—CH₂—O—CH₂—CH₂—, or the like.

A group formed by combining R_{5c} and R_{6c}, or R_{5c} and Rₓ is preferably a single bond or an alkylene group. The alkylene group may be a methylene group, an ethylene group, or the like.

Next, the cation (ZaI-4b) is described.

The cation (ZaI-4b) is a cation represented by the following formula (ZaI-4b).

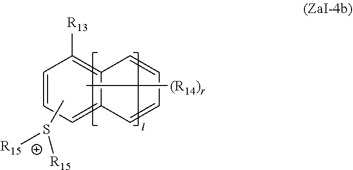

In the formula (ZaI-4b), 1 denotes an integer in the range of 0 to 2, and r denotes an integer in the range of 0 to 8.

R₁₃ denotes a hydrogen atom, a halogen atom (for example, a fluorine atom or an iodine atom), a hydroxy group, an alkyl group, an alkyl halide group, an alkoxy group, a carboxy group, an alkoxycarbonyl group, or a group containing a cycloalkyl group (which may be a cycloalkyl group itself or may be a group partially containing a cycloalkyl group). These groups may have a substituent.

R₁₄ denotes a hydroxy group, a halogen atom (for example, a fluorine atom or an iodine atom), an alkyl group, an alkyl halide group, an alkoxy group, an alkoxycarbonyl group, an alkylcarbonyl group, an alkylsulfonyl group, a cycloalkylsulfonyl group, or a group containing a cycloalkyl group (which may be a cycloalkyl group itself or may be a group partially containing a cycloalkyl group). These groups may have a substituent. A plurality of R₁₄s, if present, may be the same or different.

R₁₅ each independently denotes an alkyl group, a cycloalkyl group, or a naphthyl group. Two R₁₅s may be bonded together to form a ring. When two R₁₅s are bonded together to form a ring, the ring skeleton may include a heteroatom, such as an oxygen atom or a nitrogen atom.

In one embodiment, preferably, two R₁₅s are alkylene groups and are bonded together to form a ring structure.

The alkyl group, the cycloalkyl group, the naphthyl group, and the ring formed by two R₁₅s bonded together may have a substituent.

In the formula (ZaI-4b), an alkyl group in R₁₃, R₁₄, and R₁₅ may be linear or branched. The number of carbon atoms in the alkyl group preferably ranges from 1 to 10. The alkyl group is preferably a methyl group, an ethyl group, a n-butyl group, or a t-butyl group.

The group denoted by R₁₃ to R₁₅ may be an acid-decomposable group.

Next, the formula (ZaII) is described.

In the formula (ZaII), R²⁰⁴ and R²⁰⁵ each independently denote an aryl group, an alkyl group, or a cycloalkyl group.

The aryl group in R²⁰⁴ and R²⁰⁵ is preferably a phenyl group or a naphthyl group, more preferably a phenyl group. The aryl group in R²⁰⁴ and R²⁰⁵ may be an aryl group with a heterocycle having an oxygen atom, a nitrogen atom, a sulfur atom, or the like. The skeleton of the aryl group with a heterocycle is, for example, pyrrole, furan, thiophene, indole, benzofuran, or benzothiophene.

The alkyl group or the cycloalkyl group in R²⁰⁴ and R²⁰⁵ is preferably a linear alkyl group with 1 to 10 carbon atoms or a branched alkyl group with 3 to 10 carbon atoms (for example, a methyl group, an ethyl group, a propyl group, a butyl group, or a pentyl group), or a cycloalkyl group with 3 to 10 carbon atoms (for example, a cyclopentyl group, a cyclohexyl group, or a norbornyl group).

The aryl group, the alkyl group, and the cycloalkyl group in R²⁰⁴ and R²⁰⁵ may each independently have a substituent. A substituent that the aryl group, the alkyl group, and the cycloalkyl group of R²⁰⁴ and R²⁰⁵ may have is, for example, an alkyl group (for example, with 1 to 15 carbon atoms), a cycloalkyl group (for example, with 3 to 15 carbon atoms), an aryl group (for example, with 6 to 15 carbon atoms), an alkoxy group (for example, with 1 to 15 carbon atoms), a halogen atom, a hydroxy group, or a phenylthio group. A substituent in R²⁰⁴ and R²⁰⁵ may be an acid-decomposable group.

Specific examples of the organic cation are shown below, but the present invention is not limited thereto.

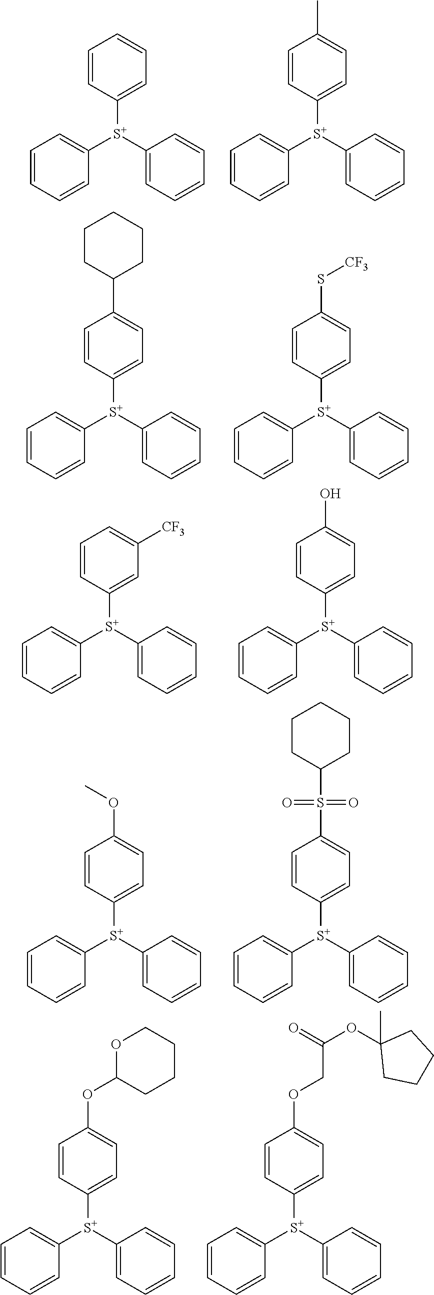

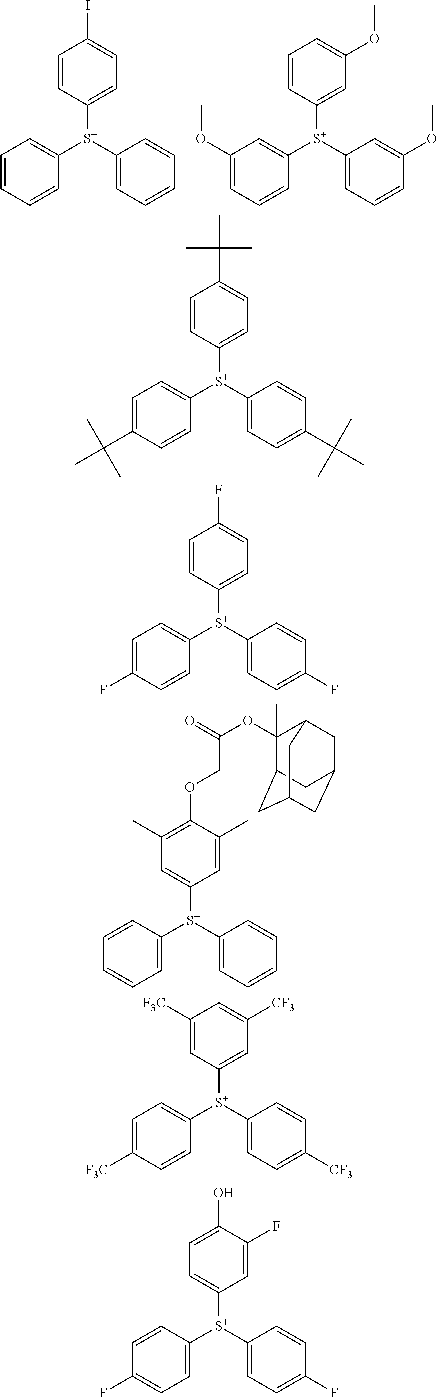

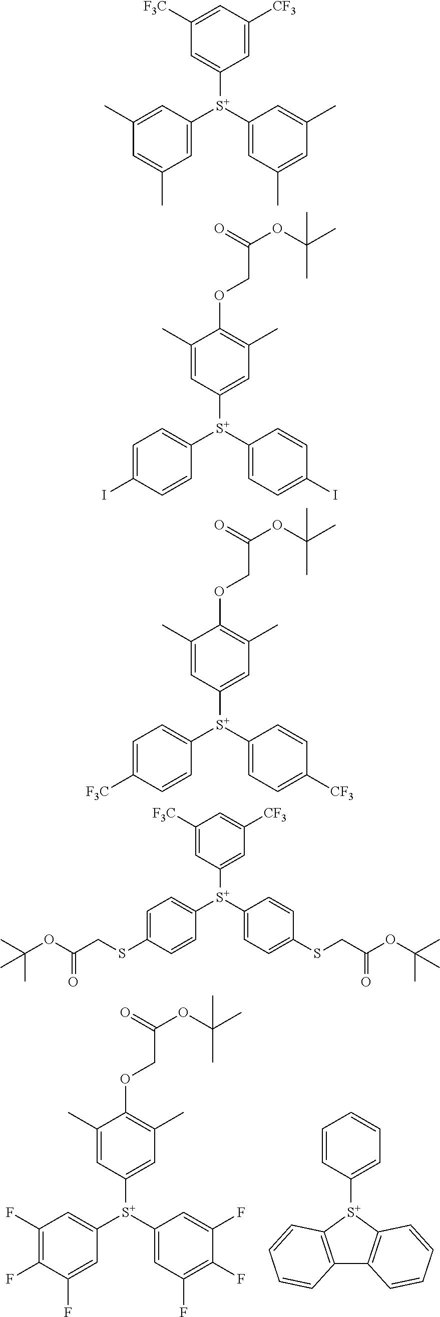

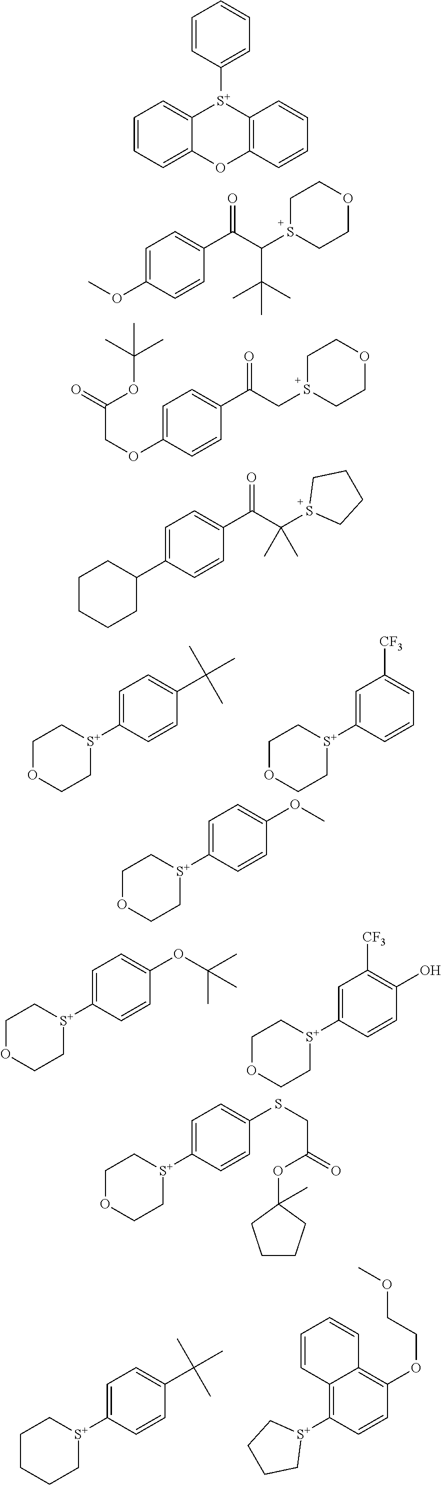

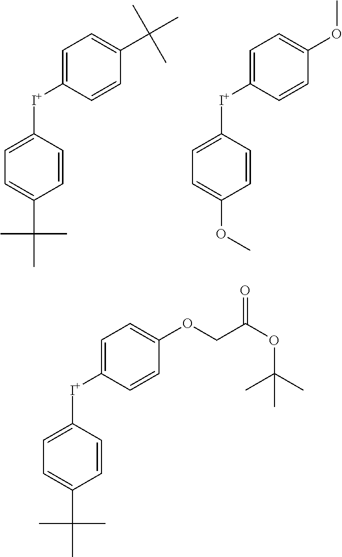 [SMILES: CC(C)(C)c1ccc([I+]c2ccc(C(C)(C)C)cc2)cc1 ; COc1ccc([I+]c2ccc(OC)cc2)cc1 ; CC(C)(C)OC(=O)COc1ccc([I+]c2ccc(C(C)(C)C)cc2)cc1]

In “M⁺X⁻”, X⁻ is an anion in which the acid dissociation constant A (pKa(A)) of an acidic compound A formed by bonding to a proton is −1.50 or less.

The acid dissociation constant A is not particularly limited as long as the requirement is satisfied, but is preferably −8.00 to −1.50, more preferably −6.00 to −1.50, still more preferably −6.00 to −1.90.

The anion is preferably an organic anion.

The organic anion is preferably an anion with an extremely low ability to cause a nucleophilic reaction, more preferably a non-nucleophilic anion.

The non-nucleophilic anion is, for example, a sulfonate anion (an aliphatic sulfonate anion, an aromatic sulfonate anion, or a camphorsulfonate anion).

The aliphatic moiety in the aliphatic sulfonate anion may be any of a linear or branched alkyl group and a cycloalkyl group, preferably a linear or branched alkyl group with 1 to 30 carbon atoms or a cycloalkyl group with 3 to 30 carbon atoms.

The alkyl group is, for example, a fluoroalkyl group (which may have a substituent other than a fluorine atom or may be a perfluoroalkyl group).

The aryl group in the aromatic sulfonate anion is preferably an aryl group with 6 to 14 carbon atoms, for example, a phenyl group, a tolyl group, or a naphthyl group.

The aromatic sulfonate anion may have a diphenyl ether structure in which phenyl groups are bonded together through an oxygen atom.

The aromatic sulfonate anion is preferably a benzenesulfonate anion, more preferably a benzenesulfonate anion substituted with a branched alkyl group or a cycloalkyl group.

The alkyl group, the cycloalkyl group, and the aryl group may have a substituent. The substituent is, for example, but not limited to, a nitro group, a halogen atom, such as a fluorine atom or a chlorine atom, a carboxy group, a hydroxy group, an amino group, a cyano group, an alkoxy group (preferably with 1 to 15 carbon atoms), an alkyl group (preferably with 1 to 10 carbon atoms), a cycloalkyl group (preferably with 3 to 15 carbon atoms), an aryl group (preferably with 6 to 14 carbon atoms), an alkoxycarbonyl group (preferably with 2 to 7 carbon atoms), an acyl group (preferably with 2 to 12 carbon atoms), an alkoxycarbonyloxy group (preferably with 2 to 7 carbon atoms), an alkylthio group (preferably with 1 to 15 carbon atoms), an alkylsulfonyl group (preferably with 1 to 15 carbon atoms), an alkyliminosulfonyl group (preferably with 1 to 15 carbon atoms), or an aryloxysulfonyl group (preferably with 6 to 20 carbon atoms).

Another non-nucleophilic anion is, for example, a fluorinated phosphorus (for example, PF₆⁻), a fluorinated boron (for example, BF₄⁻), or a fluorinated antimony (for example, SbF₆⁻).

The non-nucleophilic anion is preferably an aliphatic sulfonate anion in which at least the α-position of sulfonic acid is substituted with a fluorine atom, or an aromatic sulfonate anion substituted with a fluorine atom or a group having a fluorine atom.

In particular, a perfluoroaliphatic sulfonate anion (preferably with 4 to 8 carbon atoms) or a benzenesulfonate anion having a fluorine atom is more preferred, and a nonafluorobutane sulfonate anion, a perfluorooctane sulfonate anion, a pentafluorobenzene sulfonate anion, or a 3,5-bis(trifluoromethyl)benzene sulfonate anion is still more preferred.

The non-nucleophilic anion is also preferably an anion represented by the following formula (AN1).

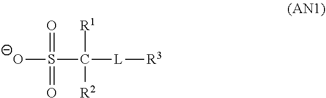

In the formula (AN1), R¹ and R₂ each independently denote a hydrogen atom or a substituent.

The substituent is preferably, but not limited to, a group that is not an electron-withdrawing group. The group that is not an electron-withdrawing group is, for example, a hydrocarbon group, a hydroxy group, an oxyhydrocarbon group, an oxycarbonyl hydrocarbon group, an amino group, a hydrocarbon-substituted amino group, or a hydrocarbon-substituted amide group.

The group that is not an electron-withdrawing group is each independently preferably —R′, —OH, —OR′, —OCOR′, —NH₂, —NR′₂, —NHR′, or —NHCOR′. R′ denotes a monovalent hydrocarbon group.

The monovalent hydrocarbon group denoted by R′ is, for example, a monovalent linear or branched hydrocarbon group, including an alkyl group, such as a methyl group, an ethyl group, a propyl group, or a butyl group; an alkenyl group, such as an ethenyl group, a propenyl group, or a butenyl group; or an alkynyl group, such as an ethynyl group, a propynyl group, or a butynyl group; a monovalent alicyclic hydrocarbon group, including a cycloalkyl group, such as a cyclopropyl group, a cyclobutyl group, a cyclopentyl group, a cyclohexyl group, a norbornyl group, or an adamantyl group; or a cycloalkenyl group, such as a cyclopropenyl group, a cyclobutenyl group, a cyclopentenyl group, or a norbornenyl group; or a monovalent aromatic hydrocarbon, including an aryl group, such as a phenyl group, a tolyl group, a xylyl group, a mesityl group, a naphthyl group, a methyl naphthyl group, an anthryl group, or a methyl anthryl group; or an aralkyl group, such as a benzyl group, a phenethyl group, a phenylpropyl group, a naphthylmethyl group, or an anthryl methyl group.

In particular, R¹ and R² preferably each independently denote a hydrocarbon group (preferably a cycloalkyl group) or a hydrogen atom.

L denotes a divalent linking group.

The divalent linking group is, for example, —O—CO—O—, —COO—, —CONH—, —CO—, —O—, —S—, —SO—, —SO₂—, an alkylene group (preferably with 1 to 6 carbon atoms), a cycloalkylene group (preferably with 3 to 15 carbon atoms), an alkenylene group (preferably with 2 to 6 carbon atoms), or a divalent linking group formed by combining a plurality of these. Among these, the divalent linking group is preferably —O—CO—O—, —COO—, —CONH—, —CO—, —O—, —SO₂—, an —O—CO—O-alkylene group-, a —COO-alkylene group-, or a —CONH-alkylene group-, more preferably —O—CO—O—, an —O—CO—O-alkylene group-, —COO—, —CONH—, —SO₂—, or a —COO— alkylene group-.

L is preferably, for example, a group represented by the following formula (AN1-1).

*^{a}—(CR^{2a}₂)_{X}-Q-(CR^{2b}₂)_{Y}—*^{b} (AN1-1)

In the formula (AN1-1), *^{a} denotes a binding position to R³ in the formula (AN1).

*^{b} denotes a binding position to —C(R¹)(R²)— in the formula (AN1).

X and Y each independently denote an integer in the range of 0 to 10, preferably 0 to 3.

R^{2a} and R^{2b} each independently denote a hydrogen atom or a substituent.

A plurality of R^{2a}s, if present, may be the same or different, and a plurality of R^{2b}s, if present, may be the same or different.

When Y is one or more, R^{2b} in CR^{2b}₂ directly bonded to —C(R¹)(R2)- in the formula (AN1) is other than a fluorine atom.

Q denotes *^{A}—O—CO—O—*^{B}, *^{A}—CO—*^{B}, *^{A}—CO—O—*^{B}, *^{A}—O—CO—*^{B}, *^{A}—O—*^{B}, *^{A}—S—*^{B} or *^{A}—SO₂—*^{B}.

However, when X+Y in the formula (AN1-1) is one or more and all of R^{2a}s and R^{2b}s in the formula (AN1-1) are hydrogen atoms, Q denotes *^{A}—O—CO—O—*^{B}, *^{A}—CO—*^{B}, *^{A}—O—CO—*^{B}, *^{A}—O—*^{B}, *^{A}—S—*^{B}, *^{A}—SO₂—*^{B}.

*^{A} denotes a binding position on the R³ side in the formula (AN1), and *^{B} denotes a binding position on the —SO₃_{^{−}} side in the formula (AN1).

R³ in the formula (AN1) denotes an organic group.

The organic group is not particularly limited as long as it has one or more carbon atoms, and may be a linear group (for example, a linear alkyl group), a branched group (for example, a branched alkyl group, such as a t-butyl group), or a cyclic group. The organic group may or may not have a substituent. The organic group may or may not have a heteroatom (such as an oxygen atom, a sulfur atom, and/or a nitrogen atom).

In particular, R³ is preferably an organic group with a ring structure. The ring structure may be monocyclic or polycyclic and may have a substituent. A ring in the organic group with a ring structure is preferably directly bonded to L in the formula (AN1).

The organic group with a ring structure, for example, may or may not have a heteroatom (such as an oxygen atom, a sulfur atom, and/or a nitrogen atom). The heteroatom may be substituted for one or more of the carbon atoms forming the ring structure.

The organic group with a ring structure is preferably, for example, a hydrocarbon group with a ring structure, a lactone ring group, or a sultone ring group. In particular, the organic group with a ring structure is preferably a hydrocarbon group with a ring structure.

The hydrocarbon group with a ring structure is preferably a monocyclic or polycyclic cycloalkyl group. These groups may have a substituent.

The cycloalkyl group may be monocyclic (such as a cyclohexyl group) or polycyclic (such as an adamantyl group), and the number of carbon atoms preferably ranges from 5 to 12.

The lactone group and the sultone group are preferably, for example, groups formed by removing one hydrogen from the ring atoms constituting the lactone structure or the sultone structure in any one of the structures represented by the formulae (LC1-1) to (LC1-21) and the structures represented by the formulae (SL1-1) to (SL1-3).

The non-nucleophilic anion is also preferably an anion represented by the following formula (AN2).

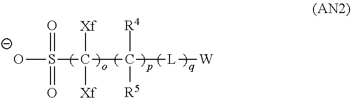

In the formula (AN2), o denotes an integer in the range of 1 to 3. p denotes an integer in the range of 0 to 10. q denotes an integer in the range of 0 to 10.

Xf each independently denotes a hydrogen atom, a fluorine atom, an alkyl group substituted with at least one fluorine atom, or an organic group having no fluorine atom. The number of carbon atoms in the alkyl group preferably ranges from 1 to 10, more preferably 1 to 4. The alkyl group substituted with at least one fluorine atom is preferably a perfluoroalkyl group.

A plurality of Xfs, if present, may be the same or different.

Xf preferably denotes a fluorine atom or a perfluoroalkyl group with 1 to 4 carbon atoms, more preferably a fluorine atom or CF₃. In particular, both Xfs are preferably fluorine atoms.

R⁴ and R⁵ each independently denote a hydrogen atom, a fluorine atom, an alkyl group, or an alkyl group substituted with at least one fluorine atom. A plurality of R⁴s, if present, may be the same or different, and a plurality of R⁵s, if present, may be the same or different.

The alkyl group preferably has 1 to 4 carbon atoms. The alkyl group may have a substituent. R⁴ and R^{s} preferably denote hydrogen atoms.

L denotes a divalent linking group. The definition of L is the same as the definition of L in the formula (AN1).

A plurality of Ls, if present, may be the same or different.

The divalent linking group is preferably-CO—, —O—, —S—, —SO—, —SO₂—, —CONH—, or a hydrocarbon group with 1 to 17 carbon atoms (for example, an alkylene group, a cycloalkylene group, or an alkenylene group).

The hydrocarbon group may have a substituent. The substituent is, for example, a halogen atom, a hydroxy group, a carboxy group, an alkyl group with 1 to 7 carbon atoms, an alkoxy group with 1 to 5 carbon atoms, an acyl group with 1 to 5 carbon atoms, an alkyloxycarbonyl group with 1 to 5 carbon atoms, or an aryl group with 6 to 8 carbon atoms. One or more methylene groups constituting the cycloalkylene group may be substituted with —O—, —S—, or —CO—.

In particular, the divalent linking group is preferably —O—, —CO—, an alkylene group, or a cycloalkylene group.

(L)_{q} is also preferably, for example, a group represented by the formula (AN2-1).

*^{a}—(Rt₁)ₓ-Q₁-((Rt₂)_{y}-Q₂)_{z}-*^{b} (AN2-1)

In the formula (AN2-1), *^{a} denotes a binding position to C(R⁴)(R⁵) in the formula (AN2). *b denotes a binding position to W in the formula (AN2).

x, y, and z each independently denote an integer in the range of 0 to 10, preferably 0 to 3, more preferably 1 to 2.

Rt₁ and Rt₂ each independently denote a divalent hydrocarbon group.

The hydrocarbon group may be an alkylene group (preferably with 1 to 7 carbon atoms), a cycloalkylene group (preferably with 3 to 17 carbon atoms), or an alkenylene group (preferably with 2 to 8 carbon atoms). The hydrocarbon group may have a substituent, and a methylene group constituting the cycloalkylene group may be substituted with —O—, —CO—, —S—, or —SO₂—.

Q₁ denotes —COO—, —CO—, —O—, —O—CO—O—, —S—, —CONH—, —SO—, or —SO₂—, preferably —COO—, —CO—, or —O—.

Q₂ denotes a single bond when y is 0, and denotes a single bond or a divalent linking group exemplified for Q₁ when y is an integer of one or more. Q₂ preferably denotes a single bond, —COO—, —CO—, or —O—.

A plurality of Rt₁s, if present, may be the same or different, a plurality of Rt₂s, if present, may be the same or different, and a plurality of Q₂s, if present, may be the same or different.
W denotes an organic group with a ring structure. In particular, a cyclic organic group is preferred.

The cyclic organic group is, for example, an alicyclic group, an aryl group, or a heterocyclic group.

The alicyclic group may be monocyclic or polycyclic. The monocyclic alicyclic group is, for example, a monocyclic cycloalkyl group, such as a cyclopentyl group, a cyclohexyl group, or a cyclooctyl group. The polycyclic alicyclic group is, for example, a polycyclic cycloalkyl group, such as a norbornyl group, a tricyclodecanyl group, a tetracyclodecanyl group, a tetracyclododecanyl group, a decahydronaphthyl group, or an adamantyl group.

In particular, an alicyclic group having a bulky structure with 7 or more carbon atoms, such as a norbornyl group, a tricyclodecanyl group, a tetracyclodecanyl group, a tetracyclododecanyl group, or an adamantyl group, is preferred.

The aryl group may be monocyclic or polycyclic. The aryl group is, for example, a phenyl group, a naphthyl group, a phenanthryl group, or an anthryl group.

The heterocyclic group may be monocyclic or polycyclic. In particular, a polycyclic heterocyclic group can further suppress the diffusion of an acid. The heterocyclic group may or may not have aromaticity. The heterocycle with aromaticity is, for example, a furan ring, a thiophene ring, a benzofuran ring, a benzothiophene ring, a dibenzofuran ring, a dibenzothiophene ring, or a pyridine ring. The heterocycle without aromaticity is, for example, a tetrahydropyran ring, a lactone ring, a sultone ring, or a decahydroisoquinoline ring. The heterocyclic group is preferably a monocyclic or polycyclic lactone ring or a monocyclic or polycyclic sultone ring.

The cyclic organic group may have a substituent. The substituent is, for example, an alkyl group (which may be linear or branched and preferably has 1 to 12 carbon atoms), a cycloalkyl group (which may be monocyclic, polycyclic, or spirocyclic and preferably has 3 to 20 carbon atoms), an aryl group (which preferably has 6 to 14 carbon atoms), a hydroxy group, an alkoxy group, an ester group, an amide group, a urethane group, a ureido group, a thioether group, a sulfonamide group, or a sulfonate group. Carbon constituting the cyclic organic group (carbon contributing to ring formation) may be a carbonyl carbon.

The anion represented by the formula (AN2) is preferably SO₃_{^{−}}—CF₂—CH₂—OCO-(L)_{q′}-W, SO₃_{^{−}}—CF₂—CHF—CH₂—OCO-(L)_{q′}-W, SO₃⁻—CF₂—COO-(L)_{q′}-W, SO₃_{^{−}}—CF₂—CF₂—CH₂—CH₂-(L)_{q}-W, or SO₃_{^{−}}—CF₂—CH(CF₃)—OCO-(L)_{q′}-W. L, q, and W are defined as in the formula (AN2). q′ denotes an integer in the range of 0 to 10.

The non-nucleophilic anion is also preferably an aromatic sulfonate anion represented by the following formula (AN3).

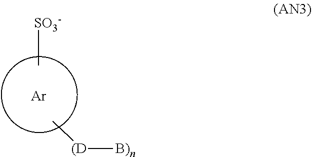

In the formula (AN3), Ar denotes an aromatic ring optionally having a substituent. The aromatic ring denoted by Ar may be monocyclic or polycyclic.

The aromatic ring denoted by Ar may be an aromatic hydrocarbon ring or a heteroaromatic ring, preferably an aromatic hydrocarbon ring.

The number of ring atoms in the aromatic ring denoted by Ar preferably ranges from 5 to 20, more preferably 6 to 12.

The aromatic ring denoted by Ar is particularly preferably a benzene ring.

An optional substituent for the aromatic ring denoted by Ar may be a halogen atom (preferably a fluorine atom) or a hydroxy group.

n denotes an integer of 0 or more. n preferably ranges from 1 to 4, more preferably 2 or 3, still more preferably 3.

D denotes a single bond or a divalent linking group. The divalent linking group may be an ether group, a thioether group, a carbonyl group, a sulfoxide group, a sulfone group, a sulfonate group, an ester group, or a group composed of two or more types thereof.

B denotes a hydrocarbon group. The hydrocarbon group denoted by B may be either an aliphatic hydrocarbon group or an aromatic hydrocarbon group.

The aliphatic hydrocarbon group denoted by B may be linear, branched, or cyclic and is preferably cyclic.

The number of carbon atoms in the aliphatic hydrocarbon group denoted by B preferably ranges from 1 to 20, more preferably 1 to 10.

In particular, the aliphatic hydrocarbon group denoted by B is preferably an alkyl group with 1 to 10 carbon atoms (a methyl group, an isopropyl group, a cyclohexyl group, a norbornyl group, or the like), more preferably a cyclic alkyl group with 1 to 10 carbon atoms.

The aromatic hydrocarbon group denoted by B may be monocyclic or polycyclic. The number of carbon atoms in the aromatic hydrocarbon group denoted by B preferably ranges from 5 to 20, more preferably 6 to 12

The aromatic hydrocarbon group denoted by B may further have a substituent. A substituent that the aromatic hydrocarbon group may have include an alkyl group with 1 to 10 carbon atoms, a halogen atom (preferably a fluorine atom), or a hydroxy group.

In particular, the aromatic hydrocarbon group denoted by B is preferably a phenyl group optionally having a substituent.

The non-nucleophilic anion may also be an anion represented by the following formula (d1-2).

Z^{2c}—SO₂^{⊖} (d 1-2)

In the formula (d1-2), Z^{2c} denotes a hydrocarbon group with 1 to 30 carbon atoms optionally having a substituent (provided that a carbon atom adjacent to S is not substituted with a fluorine atom or a perfluoroalkyl group).

The hydrocarbon group in Z^{2c} may be linear or branched and may have a ring structure. A carbon atom (preferably a carbon atom that is a ring atom when the hydrocarbon group has a ring structure) in the hydrocarbon group may be a carbonyl carbon. The hydrocarbon group is, for example, a group having a norbornyl group optionally having a substituent. A carbon atom forming the norbornyl group may be a carbonyl carbon.

“Z^{2c}—SO₃— in the formula (d1-2) is preferably different from the anions represented by the formulae (AN1) to (AN3). For example, Z^{2c} is preferably other than an aryl group. For example, atoms at the α-position and the β-position with respect to —SO₃_{^{−}} in Z^{2c} are preferably atoms other than a carbon atom having a fluorine atom as a substituent. For example, in Z^{2c}, an atom at the α-position and/or an atom at the β-position with respect to —SO₃_{^{−}} is preferably a ring atom in a cyclic group.

The photoacid generator content preferably ranges from 0.5% to 50.0% by mass, more preferably 1.0% to 30.0% by mass, still more preferably 1.0% to 25.0% by mass, based on the total solid content of the resist composition, from the perspective that the cross-sectional shape of a pattern to be formed becomes more rectangular.

The photoacid generator may be used alone or in combination of two or more types thereof. When the resist composition contains two or more photoacid generators, the total content thereof is preferably within the above range.

### [Acid Diffusion Control Agent]

A resist composition according to the present invention contains at least two acid diffusion control agents: a first acid diffusion control agent and a second acid diffusion control agent.

The acid diffusion control agent is composed of a cation and an anion and is a compound in which an acid dissociation constant (pKa(Q)) of an acidic compound produced by replacing the cation of the acid diffusion control agent with a proton is more than −1.50. In other words, the acid diffusion control agent is an onium salt that generates an acid that is a weak acid with respect to the acid generated from the photoacid generator.

When such an acid diffusion control agent collides with the acid generated from the photoacid generator, the acid generated from the photoacid generator is exchanged with the cation of the acid diffusion control agent, and a weak acid with a lower catalytic activity is released. By this reaction, the acid diffusion control agent apparently acts as a quencher that traps the acid generated from the photoacid generator or the like and suppresses the reaction between the acid in the unexposed portion and the resin.

The first acid diffusion control agent and the second acid diffusion control agent are described in detail below.

### <First Acid Diffusion Control Agent>

The first acid diffusion control agent is an acid diffusion control agent composed of a first cation and a first anion.

### (First Cation)

The first cation is a cation with a C log P value in the range of 5.400 to 6.000.

In the formation of a resist pattern, from the perspective that the first cation becomes moderately hydrophilic and diffuses into the hydrophilic exposed portion, and the acid is therefore easily quenched at the boundary between the exposed portion and the unexposed portion, the first cation more preferably has a C log P value in the range of 5.600 to 6.000.

The first cation may be any organic cation with a C log P value in the above range, for example, a sulfonium cation, an ammonium cation, or an iodonium cation. In particular, the first cation is preferably a sulfonium cation.

The organic cation is, for example, an organic cation that can be contained in the photoacid generator.

When the first cation is a sulfonium cation, the first cation is preferably the cation (ZaI), more preferably the cation (ZaI-1) or the cation (ZaI-3b), still more preferably the cation (ZaI-1).

When the first cation is an iodonium cation, the first cation is preferably the cation (ZaII).

When the first cation is a sulfonium cation, the first cation is preferably a cation selected from the group consisting of a cation represented by the formula (Q-1) to a cation represented by the formula (Q-4).

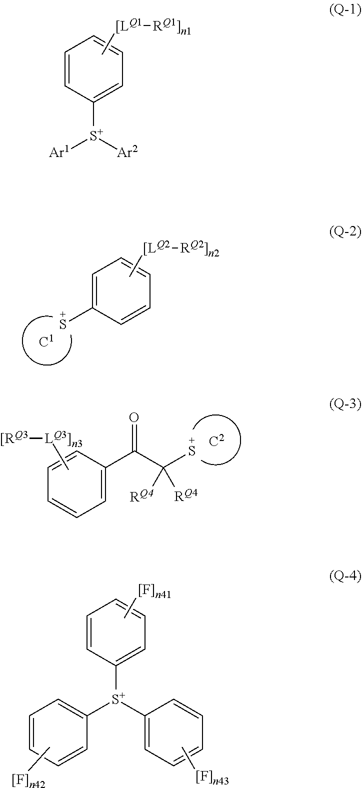

In the formula (Q-1), L^{Q1} denotes a single bond or a divalent linking group. When n1 is two or more, a plurality of L^{Q1}s may be the same or different.

The divalent linking group denoted by L^{Q1} may be —CO—, —O—, —S—, —SO—, —SO₂—, or a linking group in which a plurality of these are linked (for example, —CO—O—).

L^{Q1} preferably denotes a single bond, —CO—O— (an ester bond), or —SO₂—.

R^{Q1} denotes an alkyl group. When n1 is two or more, a plurality of RQ's may be the same or different.

The number of carbon atoms in the alkyl group denoted by R^{Q1} preferably ranges from 1 to 10, preferably 1 to 6, more preferably 1 to 3.

The alkyl group denoted by R^{Q1} may be linear, branched, or cyclic and is preferably linear or cyclic.

The alkyl group denoted by R^{Q1} may have an ethereal oxygen atom between carbon-carbon bonds. When the alkyl group is cyclic, the alkyl group may have an ethereal oxygen atom as a ring atom.

The alkyl group is preferably a methyl group, an ethyl group, a cyclohexyl group, or a 2-tetrahydropyranyl group.

n1 denotes an integer in the range of 1 to 3. n1 is preferably 1 or 2, more preferably 1.

Ar¹ and Ar² each independently denote an aryl group.

The aryl group denoted by Ar¹ and Ar² is, for example, a phenyl group or a naphthyl group, preferably a phenyl group.

Ar¹ and Ar² may be bonded together via a single bond or —O—.

In the formula (Q-2), C¹ denotes an alicyclic structure having 4 or 5 carbon atoms and including a sulfonium cation moiety.

LQ₂ denotes a single bond or a divalent linking group. When n2 is two or more, a plurality of LQ₂s may be the same or different.

The divalent linking group denoted by LQ₂ may be —CO—, —O—, —S—, —SO—, —SO₂—, or a linking group in which a plurality of these are linked.

LQ₂ preferably denotes a single bond.

RQ₂ denotes an alkyl group. When n2 is two or more, a plurality of RQ₂s may be the same or different.

The number of carbon atoms in the alkyl group denoted by RQ₂ preferably ranges from 1 to 10, preferably 1 to 6, more preferably 1 to 4.

The alkyl group denoted by RQ₂ may be linear, branched, or cyclic and is preferably linear or branched.

The alkyl group is preferably a t-butyl group.

n2 denotes an integer in the range of 1 to 3. n2 is preferably 1 or 2, more preferably 1.

In the formula (Q-3), C² denotes an alicyclic structure having 4 or 5 carbon atoms and including a sulfonium cation moiety.

L^{Q3} denotes a single bond or a divalent linking group. When n3 is two or more, a plurality of L^{Q3} may be the same or different.

The divalent linking group denoted by L^{Q3} may be —CO—, —O—, —S—, —SO—, —SO₂—, or a linking group in which a plurality of these are linked.

L^{Q3} preferably denotes a single bond.

R^{Q3} denotes an alkyl group. When n3 is two or more, a plurality of R^{Q3}s may be the same or different.

The number of carbon atoms in the alkyl group denoted by R^{Q3} preferably ranges from 1 to 10, more preferably 1 to 6.

The alkyl group denoted by R^{Q3} may be linear, branched, or cyclic and is preferably cyclic.

The alkyl group is preferably a cyclohexyl group.

n3 denotes an integer in the range of 1 to 3. n3 is preferably 1 or 2, more preferably 1.

R^{Q4} each independently denotes an alkyl group.

The number of carbon atoms in the alkyl group denoted by R^{Q4} preferably ranges from 1 to 5, more preferably 1 or 2.

The alkyl group denoted by R^{Q4} may be linear, branched, or cyclic.

In the formula (Q-4), n41, n42, and n43 each independently denote 0 or 1, and the total of n41, n42, and n43 ranges from 1 to 3.

The total of n41, n42, and n43 is preferably 1 or 3.

When the first cation is an iodonium cation, the first cation is preferably a cation represented by the formula (Q-5).

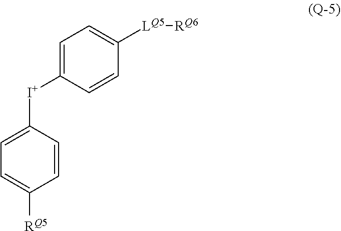

In the formula (Q-5), L^{Q5} denotes a single bond or a divalent linking group.

The divalent linking group denoted by L^{Q5} may be an alkylene group, —CO—, —O—, —S—, —SO—, —SO₂—, and a linking group in which a plurality of these are linked (for example, an O-alkylene group-CO—O—).

The alkylene group may be linear, branched, or cyclic and is preferably linear. The number of carbon atoms in the alkylene group preferably ranges from 1 to 10, more preferably 1 to 6, still more preferably 1 or 2.

R^{Q5} and R^{Q6} each independently denote an alkyl group.

The number of carbon atoms in the alkyl group preferably ranges from 1 to 10, preferably 1 to 6, more preferably 1 to 4.

The alkyl group may be linear, branched, or cyclic and is preferably branched.

The alkyl group is preferably a t-butyl group.

### (First Anion)

The first anion may be of any type as long as the acid dissociation constant B (the acid dissociation constant of an acid compound composed of the first anion and a proton) is more than −1.50.

The first anion is preferably an organic anion.

The organic anion is preferably an anion with an extremely low ability to cause a nucleophilic reaction, more preferably a non-nucleophilic anion.

An acidic compound B formed by bonding the first anion and a proton may have any pKa(B) higher than −1.50, preferably higher than pKa(A) by 0.50 or more, more preferably by 1.00 or more, still more preferably by 2.00 or more. The upper limit of the difference between pKa(B) and pKa(A) (pKa(B)-pKa(A)) is preferably, but not limited to, 15.00 or less, more preferably 10.00 or less.

pKa(B) is preferably −1.00 or more, more preferably 0.00 or more, still more preferably 0.50 or more.

The upper limit is preferably, but not limited to, 10.00 or less, more preferably 8.00 or less, still more preferably 6.00 or less.

The first anion is, for example, a sulfonate anion, a carboxylate anion, a sulfonylimide anion, a bis(alkylsulfonyl)imide anion, or a tris(alkylsulfonyl)methide anion, and from the perspective that the relationship between pKa(A) and pKa(Q) can be appropriately adjusted, a carboxylate anion, a sulfonylimide anion, a bis(alkylsulfonyl)imide anion, or a tris(alkylsulfonyl)methide anion is preferred, and a carboxylate anion is more preferred.

The first anion is also preferably an anion containing at least one selected from the group consisting of a fluorine atom and an iodine atom (more preferably a fluorine atom).

When the first anion includes a fluorine atom, the number of fluorine atoms preferably ranges from 1 to 10, more preferably 1 to 7.

The first anion is preferably an anion represented by the formula (C-1), an anion represented by the formula (C-2), or an anion represented by the formula (C-3).

R^{C1}—COO⁻ Formula (C-1)

R^{C2}-L^{C1}-COO⁻ Formula (C-2)

R^{C3}—CO—N⁻SO₂-L^{C2}-R^{C4} Formula (C-3)

In the formula (C-1), R^{C1} denotes an aryl group optionally having a substituent or an alkyl group optionally having a substituent.

The number of ring atoms in the aromatic ring constituting the aryl group denoted by R^{C1} preferably ranges from 5 to 20, more preferably 5 to 10.

The aryl group may be a phenyl group or a naphthyl group and is preferably a phenyl group.

When the aryl group has a substituent, the number of substituents in the aryl group preferably ranges from 1 to 3, more preferably 1 or 2.

A substituent that the aryl group may have may be, but is not limited to, a hydroxy group or an alkyl group optionally having a halogen atom.

The number of carbon atoms in the alkyl group optionally having a halogen atom preferably ranges from 1 to 3, more preferably 1.

The halogen atom that the alkyl group may have is preferably a fluorine atom.

The alkyl group optionally having a halogen atom may be a perfluoroalkyl group.

The alkyl group denoted by R^{C1} may be linear, branched, or cyclic and is preferably a linear alkyl group.

The number of carbon atoms in the alkyl group preferably ranges from 1 to 10, more preferably 1 to 5, still more preferably 1 to 3.

When the alkyl group has a substituent, the number of substituents in the alkyl group preferably ranges from 1 to 10, more preferably 1 to 7.

A substituent that the alkyl group may have may be, but is not limited to, a hydroxy group or a halogen atom (preferably a fluorine atom).

In the formula (C-2), R^{C2} denotes a heterocyclic group.

The heterocyclic group may be a heteroaromatic ring or a non-aromatic heterocycle and is preferably a non-aromatic heterocycle.

The number of ring atoms in the non-aromatic heterocycle preferably ranges from 4 to 8, more preferably 4 to 6.

L^{C1} denotes a single bond or a methylene group.

In the formula (C-3), R^{C3} denotes an alkyl group optionally having a substituent.

The alkyl group may be linear, branched, or cyclic and is preferably a linear or branched alkyl group.

The number of carbon atoms in the alkyl group denoted by R^{C3} preferably ranges from 1 to 10, more preferably 1 to 5, still more preferably 1 to 3.

When the alkyl group has a substituent, the number of substituents in the alkyl group preferably ranges from 1 to 10, more preferably 1 to 7.

A substituent that the alkyl group may have may be, but is not limited to, a hydroxy group or a halogen atom (preferably a fluorine atom).

L^{C2} denotes a single bond or a methylene group.

R^{C4} denotes an alicyclic hydrocarbon ring, and a methylene group in the alicyclic hydrocarbon ring may be substituted with —O—, —CO—, —S—, or —SO₂—.

The alicyclic hydrocarbon ring may be monocyclic or polycyclic and is preferably polycyclic.

The number of carbon atoms in the alicyclic hydrocarbon ring preferably ranges from 1 to 20, more preferably 1 to 10.

The alicyclic hydrocarbon ring is, for example, an adamantane ring or a norbornane ring. A methylene group in the adamantane ring and the norbornane ring may be substituted with —O—, —CO—, —S—, or —SO₂—.

The first acid diffusion control agent content preferably ranges from 0.1% to 15.0% by mass, more preferably 1.0% to 15.0% by mass, based on the total solid content of the resist composition, from the perspective that the cross-sectional shape of a pattern to be formed becomes more rectangular.

The first acid diffusion control agent may be used alone or in combination of two or more types thereof. When the resist composition contains two or more first acid diffusion control agents, the total content thereof is preferably within the above range.

From the perspective of enhancing the advantages of the present invention, the first acid diffusion control agent content is preferably 15% by mole or less, more preferably 12% by mole or less, still more preferably 10% by mole or less, based on the total number of moles of the photoacid generator, the first acid diffusion control agent, and the second acid diffusion control agent.

The lower limit is preferably, but not limited to, 0.1% by mole or more.

### <Second Acid Diffusion Control Agent>

The second acid diffusion control agent is an acid diffusion control agent composed of a second cation and a second anion.

### (Second Cation)

The second cation is an organic cation with a C log P value of 8.000 or more.

The upper limit of the C log P value is preferably, but not limited to, 15.000 or less.

The second cation may be any organic cation with a C log P value in the above range, for example, a sulfonium cation, an ammonium cation, or an iodonium cation. In particular, the second cation is preferably a sulfonium cation.

The second cation is also preferably a cation containing at least one selected from the group consisting of a fluorine atom and an iodine atom (more preferably a fluorine atom).

The organic cation is, for example, an organic cation that can be contained in the photoacid generator.

When the second cation is a sulfonium cation, the second cation is preferably the cation (ZaI), more preferably the cation (ZaI-1).

When the second cation is an iodonium cation, the second cation is preferably the cation (ZaII).

In particular, the second cation is preferably a cation represented by the formula (Q-6).

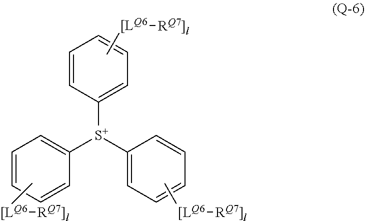

In the formula (Q-6), L^{Q6} each independently denotes a single bond or a divalent linking group.

The divalent linking group denoted by L^{Q6} may be an alkylene group with 1 to 10 carbon atoms, —CO—, —O—, —S—, —SO—, —SO₂—, or a linking group in which a plurality of these are linked.

The divalent linking group denoted by L^{Q6} is preferably a single bond, —O—CH₂—COO—, or —S—CH₂—COO—.

R^{Q7} each independently denotes an alkyl group optionally having a substituent or a halogen atom.

The alkyl group denoted by R^{Q7} may be linear, branched, or cyclic.

The number of carbon atoms in the alkyl group preferably ranges from 1 to 10, more preferably 1 to 4.

When the alkyl group has a substituent, the number of substituents in the alkyl group preferably ranges from 1 to 10, more preferably 1 to 7.

A substituent that the alkyl group may have may be, but is not limited to, a hydroxy group or a halogen atom (preferably a fluorine atom). R^{Q7} may be a fluoroalkyl group in which one or more hydrogen atoms in the alkyl group are substituted with a fluorine atom, and is preferably a perfluoroalkyl group.

The alkyl group is preferably a methyl group, an ethyl group, a t-butyl group, a trifluoromethyl group, a group formed by removing a hydrogen atom at the 2-position of adamantane, or a group formed by removing a hydrogen atom at the 2-position of 2-methyladamantane.

1 each independently denotes an integer in the range of 1 to 5.

1 preferably ranges from 1 to 3, more preferably 1 or 2.

### (Second Anion)

The second anion may be of any type as long as the acid dissociation constant C (the acid dissociation constant of an acid compound composed of the second anion and a proton) is more than −1.50.

The second anion is preferably an organic anion.

The organic anion is preferably an anion with an extremely low ability to cause a nucleophilic reaction, more preferably a non-nucleophilic anion.

An acidic compound C formed by bonding the second anion and a proton may have any pKa(C) higher than −1.50, preferably higher than pKa(A) by 0.50 or more, more preferably by 1.00 or more, still more preferably by 2.00 or more. The upper limit of the difference between pKa(C) and pKa(A) (pKa(C)-pKa(A)) is preferably, but not limited to, 15.00 or less, more preferably 10.00 or less.

pKa(C) is preferably −1.00 or more, more preferably 0.00 or more, still more preferably 0.50 or more.

The upper limit is preferably, but not limited to, 10.00 or less, more preferably 8.00 or less, still more preferably 6.00 or less.

Specific embodiments and preferred embodiments of the second anion are the same as the specific embodiments and preferred embodiments of the first anion.

The second acid diffusion control agent content preferably ranges from 0.1% to 50.0% by mass, more preferably 1.0% to 50.0% by mass, still more preferably 1.0% to 20.0% by mass, based on the total solid content of the resist composition, from the perspective that the cross-sectional shape of a pattern to be formed becomes more rectangular.

The second acid diffusion control agent may be used alone or in combination of two or more types thereof. When the resist composition contains two or more second acid diffusion control agents, the total content thereof is preferably within the above range.

The resist composition may contain an acid diffusion control agent other than those described above.

The acid diffusion control agent other than those described above is, for example, a basic compound, a low-molecular-weight compound having a nitrogen atom and having a group that leaves by the action of an acid, a compound whose acid diffusion control ability is reduced or lost by irradiation with an actinic ray or radiation, or the like.

For example, compounds described in paragraphs [0132] to [0164] of WO2020/066824A and known compounds disclosed in paragraphs [0627] to [0664] of US2016/0070167A1, paragraphs [0095] to [0187] of US2015/0004544A1, paragraphs [0403] to [0423] of US2016/0237190A1, and paragraphs [0259] to [0328] of US2016/0274458A1 can be used.

The one or more photoacid generators may be bonded to the first acid diffusion control agent or the second acid diffusion control agent via a covalent bond. A compound produced by bonding the photoacid generator and the first acid diffusion control agent or the second acid diffusion control agent via a covalent bond is hereinafter referred to as a “bound product”.

The bound product may be a compound produced by bonding the photoacid generator and the first acid diffusion control agent via a covalent bond or may be a compound produced by bonding the photoacid generator and the second acid diffusion control agent via a covalent bond. As described later, the bound product may be a compound produced by bonding two or more photoacid generators and the first acid diffusion control agent via a covalent bond or may be a compound produced by bonding two or more photoacid generators and the second acid diffusion control agent via a covalent bond.

The bound product in the resist composition may be only one type or two or more types. The resist composition may contain, in addition to the bound product, another photoacid generator that does not form a bound product or another acid diffusion control agent that does not form a bound product.

When the resist composition includes the bound product, the following embodiments 1 to 3 are preferred from the perspective of enhancing the advantages of the present invention.

Embodiment 1: An embodiment including a first bound product produced by bonding two photoacid generators and any one acid diffusion control agent of the first acid diffusion control agent and the second acid diffusion control agent (hereinafter also referred to as an acid diffusion control agent X) via a covalent bond, a second bound product produced by bonding one photoacid generator and the acid diffusion control agent X via a covalent bond, and the other acid diffusion control agent of the first acid diffusion control agent and the second acid diffusion control agent.
Embodiment 2: An embodiment including a bound product produced by bonding one photoacid generator and any one of the first acid diffusion control agent and the second acid diffusion control agent via a covalent bond, the other acid diffusion control agent of the first acid diffusion control agent and the second acid diffusion control agent, and a photoacid generator that is a compound different from the bound product.
Embodiment 3: An embodiment including a bound product produced by bonding one photoacid generator and any one of the first acid diffusion control agent and the second acid diffusion control agent via a covalent bond, and the other acid diffusion control agent of the first acid diffusion control agent and the second acid diffusion control agent.

When bonded via a covalent bond, the photoacid generator and the first acid diffusion control agent may be bonded via a single bond or a divalent linking group. The divalent linking group may be —CO—, —O—, —S—, —SO—, —SO₂—, a divalent hydrocarbon group (for example, an alkylene group, a cycloalkylene group, an alkenylene group, an arylene group, or the like), or a linking group in which a plurality of these groups are linked. The divalent hydrocarbon group may have a fluorine atom or an iodine atom as a substituent.

When the photoacid generator and the first acid diffusion control agent are bonded via a covalent bond, an anion in the photoacid generator and a first anion in the first acid diffusion control agent may be bonded via a covalent bond, or a cation in the photoacid generator and a first cation in the first acid diffusion control agent may be bonded via a covalent bond. From the perspective of the ease of synthesis, a bound product produced by bonding an anion in the photoacid generator and a first anion in the first acid diffusion control agent via a covalent bond is preferred.

An example of an embodiment in which the photoacid generator and the first acid diffusion control agent are covalently bonded together may be an embodiment in which a residue formed by removing one atom in the photoacid generator and a residue formed by removing one atom in the first acid diffusion control agent are bonded together via a single bond or a divalent linking group.

When the photoacid generator and the first acid diffusion control agent are bonded via a covalent bond, two photoacid generators and one first acid diffusion control agent may be bonded via a covalent bond. When two photoacid generators and one first acid diffusion control agent are bonded via a covalent bond, an anion in the two photoacid generators and a first anion in the first acid diffusion control agent may be bonded via a covalent bond, or a cation in the two photoacid generators and a first cation in the first acid diffusion control agent may be bonded via a covalent bond.

When bonded via a covalent bond, the photoacid generator and the second acid diffusion control agent may be bonded via a single bond or a divalent linking group. The divalent linking group may be —CO—, —O—, —S—, —SO—, —SO₂—, a divalent hydrocarbon group (for example, an alkylene group, a cycloalkylene group, an alkenylene group, an arylene group, or the like), or a linking group in which a plurality of these groups are linked. The divalent hydrocarbon group may have a fluorine atom or an iodine atom as a substituent.

When the photoacid generator and the second acid diffusion control agent are bonded via a covalent bond, an anion in the photoacid generator and a second anion in the second acid diffusion control agent may be bonded via a covalent bond, or a cation in the photoacid generator and a second cation in the second acid diffusion control agent may be bonded via a covalent bond. From the perspective of the ease of synthesis, a bound product produced by bonding an anion in the photoacid generator and a second anion in the second acid diffusion control agent via a covalent bond is preferred.

An example of an embodiment in which the photoacid generator and the second acid diffusion control agent are covalently bonded together may be an embodiment in which a residue formed by removing one atom in the photoacid generator and a residue formed by removing one atom in the second acid diffusion control agent are bonded together via a single bond or a divalent linking group.

When the photoacid generator and the second acid diffusion control agent are bonded via a covalent bond, two photoacid generators and one second acid diffusion control agent may be bonded via a covalent bond. When two photoacid generators and one second acid diffusion control agent are bonded via a covalent bond, an anion in the two photoacid generators and a second anion in the second acid diffusion control agent may be bonded via a covalent bond, or a cation in the two photoacid generators and a second cation in the second acid diffusion control agent may be bonded via a covalent bond.

The bound product content preferably ranges from 0.1% to 50.0% by mass, more preferably 1.0% to 40.0% by mass, still more preferably 1.0% to 35.0% by mass based on the total solid content of the resist composition.

### [Hydrophobic Resin]

The resist composition may contain a hydrophobic resin different from the specific resin.

The hydrophobic resin is preferably designed to be unevenly distributed on the surface of a resist film, but unlike surfactants it does not necessarily need to have a hydrophilic group in the molecule and may not contribute to uniform mixing of a polar substance and a nonpolar substance.

The effects of adding the hydrophobic resin may be the control of the static and dynamic contact angles of a resist film surface with respect to water and may be the suppression of outgassing.

From the perspective of uneven distribution in the film surface layer, the hydrophobic resin preferably has any one or more, more preferably two or more, of a fluorine atom, a silicon atom, and a CH₃ partial structure included in a side chain portion of the resin.

The hydrophobic resin preferably has a hydrocarbon group with 5 or more carbon atoms. These groups may be present in the main chain of the resin or may be substituted on the side chain.

The hydrophobic resin may be a compound described in paragraphs [0275] to [0279] of WO2020/004306A.

The hydrophobic resin may be used alone or in combination of two or more types thereof.

When the resist composition contains a hydrophobic resin, the hydrophobic resin content preferably ranges from 0.01% to 20.0% by mass, more preferably 0.1% to 15.0% by mass, based on the total solid content of the resist composition.

### [Surfactant]

The resist composition may contain a surfactant.

A surfactant, if present, improves the adhesiveness and allows a pattern with fewer development defects to be formed.

The surfactant is preferably a fluorinated and/or silicon-based surfactant, more preferably a silicon-based surfactant from the perspective of environmental regulations.

The fluorinated and/or silicon-based surfactant is, for example, a surfactant described in paragraphs [0218] and [0219] of WO2018/193954A.

The surfactant may be used alone or in combination of two or more types thereof.

When the resist composition contains a surfactant, the surfactant content preferably ranges from 0.0001% to 2.0% by mass, more preferably 0.0005% to 1.0% by mass, still more preferably 0.1% to 1.0% by mass, based on the total solid content of the resist composition.

### [Solvent]

The resist composition preferably contains a solvent.

The solvent preferably contains (CP) a propylene glycol monoalkyl ether carboxylate and (M2) at least one selected from the group consisting of a propylene glycol monoalkyl ether, a lactate, an acetate, an alkoxypropionate, a chain ketone, a cyclic ketone, a lactone, and an alkylene carbonate.

A combination of the solvent and the specific resin is preferred from the perspective of improving the coating performance of the resist composition and reducing the number of development defects of a pattern. Due to a good balance between the solubility of the resin, the boiling point, and the viscosity, the solvent can suppress the unevenness of the thickness of a resist film, the generation of precipitates during spin coating, and the like.

The details of the component (CP) and the component (M2) are described in paragraphs [0218] to [0226] of WO2020/004306A, the contents of which are incorporated herein.

The solvent may further contain a component other than the component (CP) and the component (M2).

When the solvent further contains a component other than the component (CP) and the component (M2), the amount of the component other than the component (CP) and the component (M2) preferably ranges from 5% to 30% by mass based on the total amount of the solvent.

From the perspective of further improving the coating performance of the resist composition, the solvent content of the resist composition preferably ranges from 70% to 95.5% by mass, more preferably 80% to 99% by mass.

### [0229][Other Additives]

The resist composition may contain an additive other than those described above.

The other additive may be a dissolution inhibiting compound (a compound with a molecular weight of 3000 or less, which is decomposed by the action of an acid to decrease the solubility in an organic-based developer), a dye, a plasticizer, a photosensitizer, a light absorber, or a compound that improves the solubility in a developer (for example, a phenolic compound with a molecular weight of 1,000 or less or an alicyclic or aliphatic compound with a carbonyl group).

The resist composition may contain water, but the water content is preferably low.

The water content often ranges from 1 to 30,000 ppm by mass, preferably 10,000 ppm by mass or less, more preferably 5,000 ppm by mass or less, still more preferably 1,000 ppm by mass or less, based on the total mass of the resist composition. The lower limit is preferably, but not limited to, 0 ppm by mass.

The resist composition may contain water, but the residual monomer content is preferably low. The residual monomer is, for example, a monomer used for the synthesis of the specific resin.

The residual monomer content often ranges from 1 to 30,000 ppm by mass, preferably 10,000 ppm by mass or less, more preferably 5,000 ppm by mass or less, still more preferably 1,000 ppm by mass or less, based on the total mass of the resist composition. The lower limit is preferably, but not limited to, 0 ppm by mass.

A resist composition in the present specification is suitably used as a resist composition for EUV exposure.

EUV light has a wavelength of 13.5 nm, which is shorter than the wavelength of ArF light (wavelength: 193 nm) or the like, and the number of incident photons is small in exposure at the same sensitivity. This results in a large influence of “photon shot noise” in which the number of photons varies stochastically, which leads to deterioration of LER and a bridge defect. To reduce the photon shot noise, there is a method of increasing the exposure level to increase the number of incident photons, but there is a trade-off with the demand for high sensitivity.

### [Pattern Forming Method]

A pattern forming method according to the present invention includes the following steps.
Step 1: A step of forming a resist film on a substrate using a resist composition
Step 2: A step of exposing the resist film
Step 3: A step of developing the exposed resist film using a developer to form a resist pattern

Each step may be performed only once or multiple times.

Each step is described in detail below.

### [Step 1: Resist Film Forming Step]

The step 1 is a step of forming a resist film on a substrate using a resist composition.

The resist composition is defined as described above.

A method of forming a resist film on a substrate using a resist composition is, for example, a method of applying a resist composition to a substrate.

If necessary, the resist composition is preferably filtered before application. The filter preferably has a pore size of 0.1 μm or less, more preferably 0.05 μm or less, still more preferably 0.03 μm or less. The filter is preferably made of polytetrafluoroethylene, polyethylene, or nylon.

The substrate can be, but is not limited to, a substrate typically used in a process of producing a semiconductor, such as an IC, a process of producing a circuit board, such as a liquid crystal or a thermal head, a lithography process of another photofabrication, or the like. Specific examples thereof include inorganic substrates, such as silicon, SiO₂, and SiN.

An underlying film (for example, an inorganic film, an organic film, or an antireflection film) may be formed under the resist film.

The resist composition can be applied, for example, to a substrate using a spinner, a coater, or the like. The application method is preferably spin coating using a spinner. The rotational speed in spin coating using a spinner preferably ranges from 1000 to 3000 rpm.

The application of the resist composition may be followed by a drying treatment to form a resist film. The drying method is, for example, a method of drying by heating. The heating can be performed using a means provided in a typical exposure apparatus and/or developing apparatus or using a hot plate or the like.

The heating temperature preferably ranges from 80° C. to 150° C., more preferably 80° C. to 140° C., still more preferably 80° C. to 130° C. The heating time preferably ranges from 30 to 1000 seconds, more preferably 60 to 800 seconds, still more preferably 60 to 600 seconds.

The thickness of the resist film is preferably, but not limited to, in the range of 10 to 120 nm from the perspective of forming a micropattern with higher accuracy. In particular, for EUV exposure, the resist film more preferably has a thickness in the range of 10 to 65 nm, still more preferably 15 to 50 nm. For ArF liquid immersion exposure, the resist film more preferably has a thickness in the range of 10 to 120 nm, still more preferably 15 to 90 nm.

A top coat may be formed on the resist film using a top coat composition.

The top coat composition is preferably a composition that is not mixed with the resist film and can be uniformly applied to an upper layer of the resist film.

The top coat preferably has a thickness in the range of 10 to 200 nm, more preferably 20 to 100 nm, still more preferably 40 to 80 nm.

The composition and the formation method of the top coat are not particularly limited, and a known top coat can be formed using a known method. For example, the top coat can be formed on the basis of the description in paragraphs [0072] to [0082] of JP2014-059543A.

For example, a top coat including a basic compound described in JP2013-061648A is preferably formed on the resist film. The top coat also preferably includes a compound including at least one group or bond selected from the group consisting of an ether bond, a thioether bond, a hydroxy group, a thiol group, a carbonyl group, and an ester group.

### [Step 2: Exposure Step]

The step 2 is a step of exposing the resist film.

The exposure method may be a method of irradiating a formed resist film with an actinic ray or radiation through a predetermined mask.

The actinic ray or radiation may be infrared light, visible light, ultraviolet light, far-ultraviolet light, extreme ultraviolet light, X-rays, or an electron beam, preferably far-ultraviolet light with a wavelength of 250 nm or less, more preferably 220 nm or less, particularly preferably 1 to 200 nm, more specifically, a KrF excimer laser (248 nm), an ArF excimer laser (193 nm), an F2 excimer laser (157 nm), EUV (13.5 nm), X-rays, or an electron beam.

The exposure is preferably followed by a post-exposure heat treatment (post exposure bake (PEB)) before development. The post-exposure heat treatment promotes a reaction in the exposed portion and improves sensitivity and the pattern shape. The heating can be performed using a means provided in a typical exposure apparatus and/or developing apparatus or using a hot plate or the like.

The heating temperature preferably ranges from 80° C. to 150° C., more preferably 80° C. to 140° C., still more preferably 80° C. to 130° C.

The heating time preferably ranges from 10 to 1000 seconds, more preferably 10 to 180 seconds, still more preferably 30 to 120 seconds.

### [Step 3: Development Step]

The step 3 is a step of developing the exposed resist film using a developer to form a resist pattern.

The developer may be an alkaline developer or a developer containing an organic solvent (hereinafter also referred to as an “organic-based developer”).

The developing method is, for example, a method of dipping a substrate in a vessel filled with the developer for a certain period (a dip method), a method of raising the developer on the surface of a substrate by surface tension and standing still for a certain period for development (a paddle method), a method of spraying the developer on the surface of a substrate (a spray method), or a method of continuously ejecting the developer while moving a developer ejection nozzle at a constant speed on a substrate rotating at a constant speed (a dynamic dispense method).

The developing step may be followed by a step of stopping the development while replacing the developer with another solvent.

The development time is preferably, but not limited to, 10 to 300 seconds, more preferably 20 to 120 seconds, provided that the resin in an unexposed portion is sufficiently dissolved.

The temperature of the developer preferably ranges from 0° C. to 50° C., more preferably 15° C. to 35° C.

The alkaline developer is preferably an alkaline aqueous solution containing an alkali. The type of the alkaline aqueous solution is, for example, an alkali solution containing a quaternary ammonium salt exemplified by tetramethylammonium hydroxide, an inorganic alkali, a primary amine, a secondary amine, a tertiary amine, an alcohol amine, a cyclic amine, or the like. In particular, the alkaline developer is preferably an aqueous solution of a quaternary ammonium salt exemplified by tetramethylammonium hydroxide (TMAH).

An appropriate amount of alcohol may be added to the alkaline developer. The concentration of a basic compound in the alkaline developer typically ranges from 0.1% to 20% by mass. The alkaline developer typically has a pH in the range of 10.0 to 15.0.

The organic-based developer preferably contains at least one selected from the group consisting of a ketone solvent, an ester solvent, an alcohol solvent, an amide solvent, an ether solvent, and a hydrocarbon solvent. The ketone solvent and the ester solvent may be organic solvents described in paragraphs [0179] and [0180] of JP2022-125078A, and the alcohol solvent, the amide solvent, the ether solvent, and the hydrocarbon solvent may be solvents disclosed in paragraphs <0715> to <0718> of US2016/0070167A1.

Organic solvents in the organic-based developer may be mixed or may be mixed with water. The moisture content of the organic-based developer as a whole is preferably less than 50% by mass, more preferably less than 20% by mass, still more preferably less than 10% by mass, and it is particularly preferable that the organic-based developer includes substantially no moisture.

The organic solvent content of the organic-based developer preferably ranges from 50% to 100% by mass, more preferably 80% to 100% by mass, still more preferably 90% to 100% by mass, particularly preferably 95% to 100% by mass, based on the total mass of the developer.

If necessary, the developer may contain a surfactant.

### [Other Steps]

The pattern forming method preferably includes a rinsing step of washing the pattern using a rinse liquid after the step 3.

The rinse liquid may be any rinse liquid that does not dissolve the pattern.

The rinse liquid used in the rinsing step after the development step using the alkaline developer is, for example, pure water.

The rinse liquid used in the rinsing step after the development step using the organic-based developer can be a solution containing a typical organic solvent that does not dissolve the pattern. The organic solvent in the rinse liquid is preferably at least one organic solvent selected from the group consisting of a hydrocarbon solvent, a ketone solvent, an ester solvent, an alcohol solvent, an amide solvent, and an ether solvent.

An appropriate amount of surfactant may be added to the rinse liquid.

The washing method is, for example, but not limited to, a method of continuously ejecting the rinse liquid to a substrate rotating at a constant speed (a spin coating method), a method of dipping a substrate in a vessel filled with the rinse liquid for a certain period (a dipping method), or a method of spraying the rinse liquid on the surface of a substrate (a spray method).

The pattern forming method may include a heating step (post bake) after the rinsing step. In this step, the developer and the rinse liquid remaining between patterns and inside patterns are removed. This step also anneals a resist pattern and improves the surface roughness of the pattern.

The heating step after the rinsing step is preferably performed at 40° C. to 250° C. (preferably 90° C. to 200° C.) for 10 seconds to 3 minutes (preferably 30 seconds to 120 seconds).

A formed pattern may be used as a mask to perform etching on a substrate to be etched. More specifically, the pattern formed in the step 3 may be used as a mask to process a substrate (or an underlayer film and the substrate) and form a pattern on the substrate.

The substrate (or the underlayer film and the substrate) may be processed by any method, preferably by a method of using the pattern formed in the step 3 as a mask to dry-etch the substrate (or the underlayer film and the substrate) and form a pattern on the substrate. The dry etching is preferably oxygen plasma etching.

Various materials used in a resist composition and in a pattern forming method according to the present invention (for example, a solvent, a developer, a rinse liquid, a composition for forming an antireflection film, a composition for forming a top coat, and the like) preferably do not include an impurity, such as metal. The impurity content of each material is preferably 1 ppm by mass or less, more preferably 10 ppb by mass or less, still more preferably 100 ppt by mass or less, particularly preferably 10 ppt by mass or less, most preferably 1 ppt by mass or less. The lower limit is preferably, but not limited to, 0 ppt by mass. The metal impurity is, for example, Na, K, Ca, Fe, Cu, Mg, Al, Li, Cr, Ni, Sn, Ag, As, Au, Ba, Cd, Co, Pb, Ti, V, W, Zn, or the like.

A method for removing an impurity, such as metal, from various materials is, for example, filtration using a filter. For the filtration using a filter, a method described in paragraph [0321] of WO2020/004306A can be used.

A method of decreasing an impurity, such as metal, in various materials is, for example, a method of selecting a raw material with a low metal content as a raw material constituting various materials, a method of filtering a raw material constituting various materials through a filter, a method of performing distillation under conditions in which contamination is suppressed as much as possible by lining the inside of an apparatus with Teflon (registered trademark), or the like.

In addition to filter filtration, an impurity may be removed using an adsorbent, or filter filtration and an adsorbent may be used in combination. The adsorbent may be a known adsorbent, for example, an inorganic adsorbent, such as silica gel or zeolite, or an organic adsorbent, such as activated carbon. To decrease an impurity, such as metal, in the various materials, it is necessary to prevent contamination with a metal impurity in the production process. Whether or not a metal impurity is sufficiently removed from a production apparatus can be confirmed by measuring the metal component content of a washing liquid used for washing the production apparatus. The metal component content of the used washing liquid preferably ranges from 100 parts per trillion (ppt) by mass or less, more preferably 10 ppt by mass or less, still more preferably 1 ppt by mass or less. The lower limit is preferably, but not limited to, 0 ppt by mass or more.

An electrically conductive compound may be added to an organic treatment liquid, such as a developer or a rinse liquid, in order to prevent a failure of chemical liquid piping and various parts (a filter, an O-ring, a tube, and the like) due to charging of static electricity and subsequent electrostatic discharge. The electrically conductive compound is, for example, but not limited to, methanol. The addition amount is preferably, but not limited to, 10% by mass or less, more preferably 5% by mass or less, from the perspective of maintaining preferred development characteristics or rinsing characteristics. The lower limit is preferably, but not limited to, 0.01% by mass or more.

The chemical liquid piping is, for example, a pipe made of stainless steel (SUS) or coated with polyethylene, polypropylene, or a fluoropolymer (such as polytetrafluoroethylene or perfluoroalkoxy resin) subjected to an antistatic treatment. Similarly, the filter and the O-ring may be made of polyethylene, polypropylene, or a fluoropolymer (such as polytetrafluoroethylene or perfluoroalkoxy resin) subjected to antistatic treatment.

### [Method for Producing Device]

The present invention also relates to a method for producing an electronic device including the pattern forming method and to an electronic device produced by the production method.

An electronic device according to the present invention is suitably mounted on electrical and electronic equipment (a home appliance, office automation (OA), media-related equipment, optical equipment, communication equipment, or the like).

### EXAMPLES

The present invention is described in more detail in the following examples.

Materials, use amounts, ratios, treatment contents, treatment procedures, and the like in the following examples can be appropriately changed without departing from the gist of the present invention. Thus, the scope of the present invention should not be construed as being limited to the examples described below.

### [Each Component of Resist Composition]

Components of resist compositions used in the examples and the comparative examples are described below.

### [Specific Resin]

Table 1 shows the type, the compositional ratio (mass ratio; in order from the left), the weight-average molecular weight (Mw), and the dispersity (Mw/Mn) of each repeating unit in resins E-1 to E-14 used as specific resins.

The weight-average molecular weight (Mw) and the dispersity (Mw/Mn) of the specific resins were measured by GPC (carrier: tetrahydrofuran (THF)) (polystyrene equivalent). The compositional ratio (% by mass) was measured by ¹³C-NMR (nuclear magnetic resonance).

The resins E-1 to E-14 were synthesized according to the synthesis method of the resin E-1 (Synthesis Example 1) described later.

TABLE 1

| | | Ratio | | Ratio of | | Ratio of | | Ratio of | | |
|---|---|---|---|---|---|---|---|---|---|---|
| | | of first | | second | | third | | fourth | | |
| | First | unit | Second | unit | Third | unit | Fourth | unit | | |
| Resin | unit | (wt %) | unit | (wt %) | unit | (wt %) | unit | (wt %) | Mw | Mw/Mn |
| E-1 | Pol-1 | 50 | Pol-2 | 50 | — | — | — | — | 6,500 | 1.52 |
| E-2 | Pol-3 | 20 | Pol-4 | 10 | Pol-5 | 70 | — | — | 7,400 | 1.55 |
| E-3 | Pol-3 | 25 | Pol-4 | 15 | Pol-5 | 60 | — | — | 12,000 | 1.46 |
| E-4 | Pol-6 | 25 | Pol-4 | 15 | Pol-5 | 60 | — | — | 6,400 | 1.76 |
| E-5 | Pol-3 | 20 | Pol-4 | 10 | Pol-7 | 70 | — | — | 7,800 | 1.47 |
| E-6 | Pol-3 | 25 | Pol-4 | 15 | Pol-8 | 60 | — | — | 7,000 | 1.61 |
| E-7 | Pol-3 | 20 | Pol-9 | 20 | Pol-5 | 60 | — | — | 6,500 | 1.57 |
| E-8 | Pol-3 | 20 | Pol-10 | 20 | Pol-5 | 60 | — | — | 6,900 | 1.87 |
| E-9 | Pol-3 | 20 | Pol-11 | 20 | Pol-5 | 60 | — | — | 8,200 | 1.84 |
| E-10 | Pol-3 | 25 | Pol-4 | 10 | Pol-5 | 60 | Pol-10 | 5 | 9,200 | 1.50 |
| E-11 | Pol-3 | 25 | Pol-4 | 10 | Pol-5 | 60 | Pol-11 | 5 | 9,100 | 1.49 |
| E-12 | Pol-12 | 30 | Pol-9 | 35 | Pol-7 | 35 | — | — | 6,500 | 1.52 |
| E-13 | Pol-12 | 35 | Pol-9 | 20 | Pol-5 | 45 | — | — | 6,900 | 1.55 |
| E-14 | Pol-13 | 30 | Pol-9 | 20 | Pol-5 | 50 | — | — | 7,230 | 1.69 |

The structures of the repeating units in the resins E-1 to E-14 shown in Table 1 are shown below.

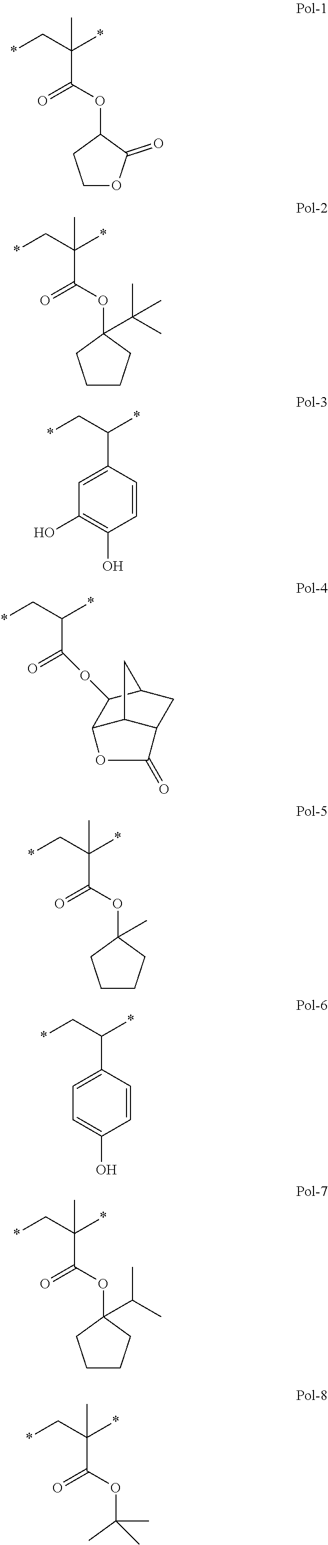

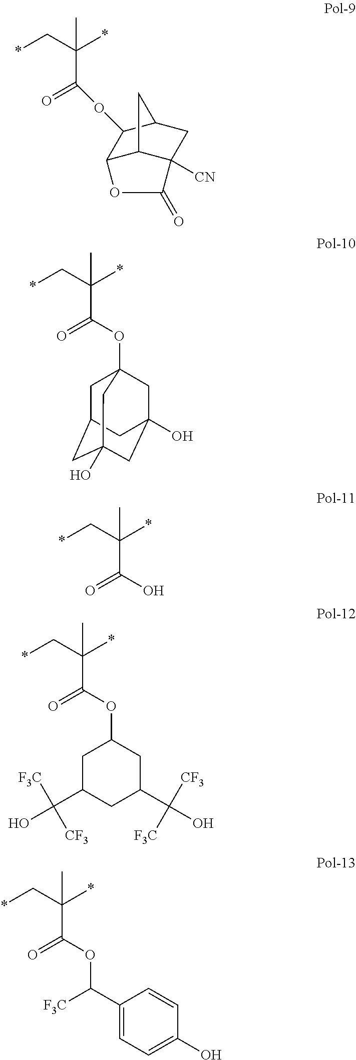

### Pol-13

The resin E-1 was synthesized according to the following scheme.

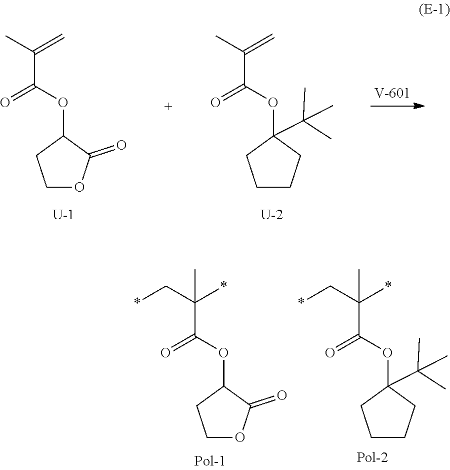

### Synthesis Example 1: Synthesis of Resin E-1

Cyclohexanone (113 g) was heated to 80° C. in a nitrogen stream. While stirring this liquid, a mixed solution of monomer U-1 (25.5 g), monomer U-2 (31.6 g), cyclohexanone (210 g), and dimethyl 2,2′-azobisisobutyrate [V-601, manufactured by Wako Pure Chemical Industries, Ltd.](6.21 g) was added dropwise over 6 hours to prepare a reaction liquid. After completion of the dropwise addition, the reaction liquid was stirred at 80° C. for another 2 hours.

The reaction liquid was cooled, was then reprecipitated with a large amount of methanol/water (mass ratio: 9:1), and was filtered, and the resultant solid was dried under vacuum to produce 52 g of the resin E-1.

The resin E-1 had a weight-average molecular weight (Mw: polystyrene equivalent) of 6500 and a dispersity (Mw/Mn) of 1.52 as determined by GPC (carrier: tetrahydrofuran (THF)). The compositional ratio measured by ¹³C-NMR (nuclear magnetic resonance) was 50/50 in mass ratio.

[Photoacid Generator] The structures of photoacid generators P-1 to P-8 are shown below.

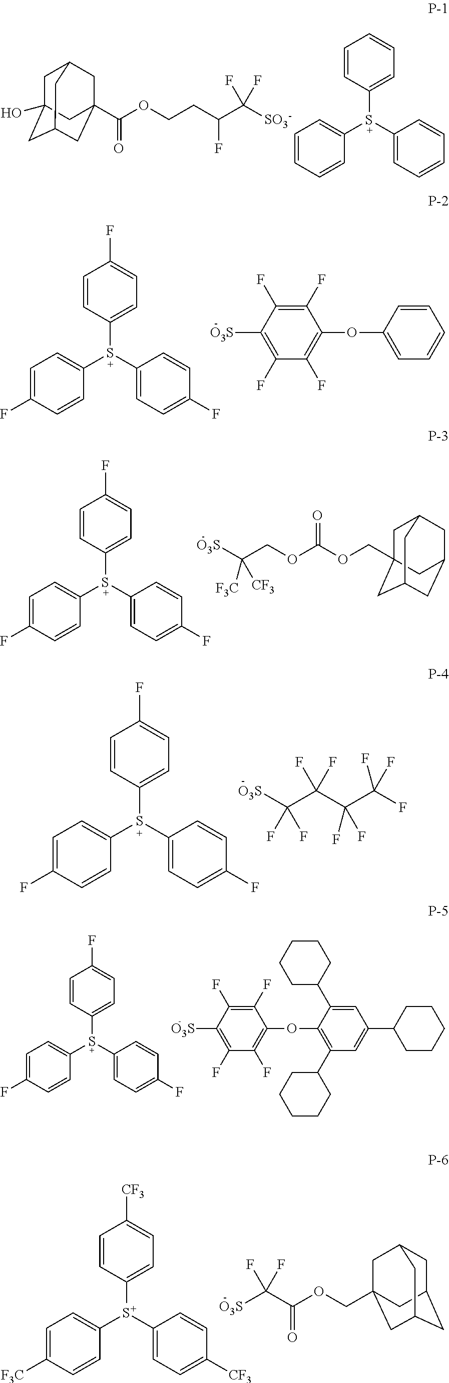

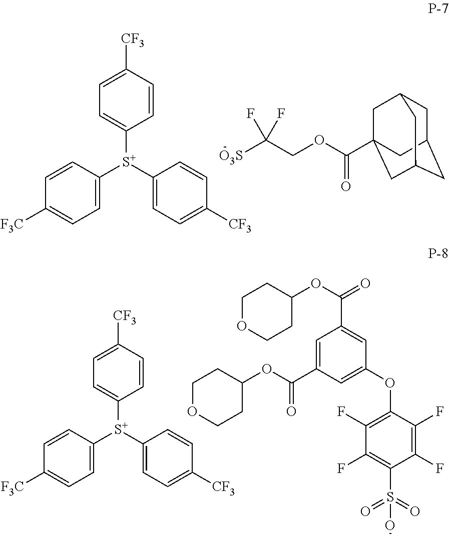

### [First Acid Diffusion Control Agent]

The structures of first cations CQ₁-4 to CQ₁-12, cations CQ₁-1 to CQ₁-3 not corresponding to the first cations, and first anions CA1-1 to CA1-9 of the first acid diffusion control agent are shown below.

The combinations of the first cation and the first anion of the first acid diffusion control agent in each of the examples and the comparative examples are shown below in Tables 2 to 4.

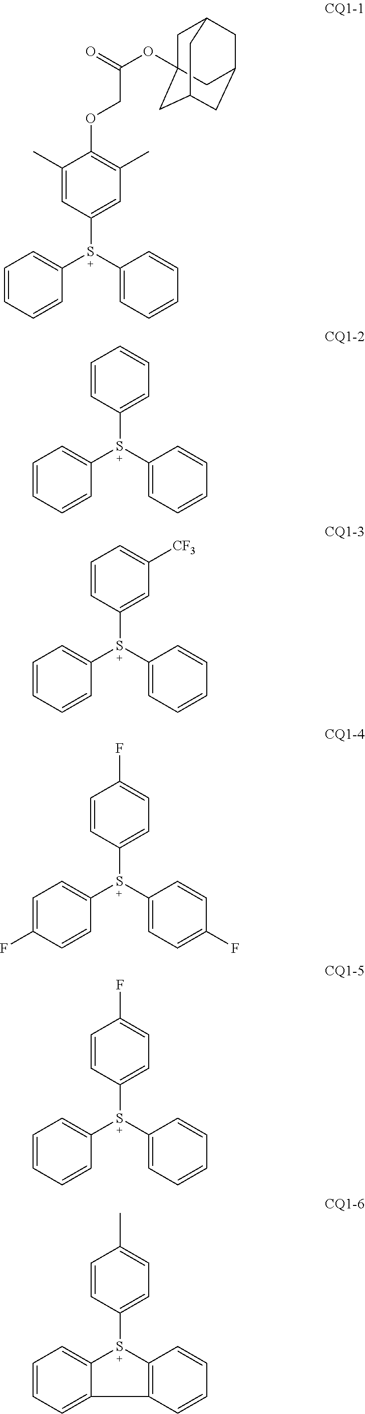

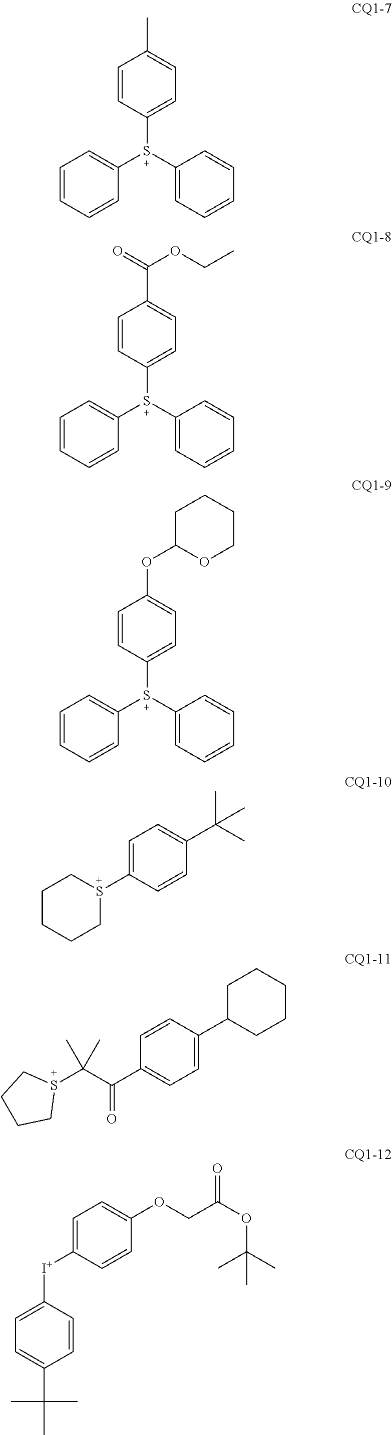

The structures of the first anions CA1-1 to CA1-9 of the first acid diffusion control agent are shown below.

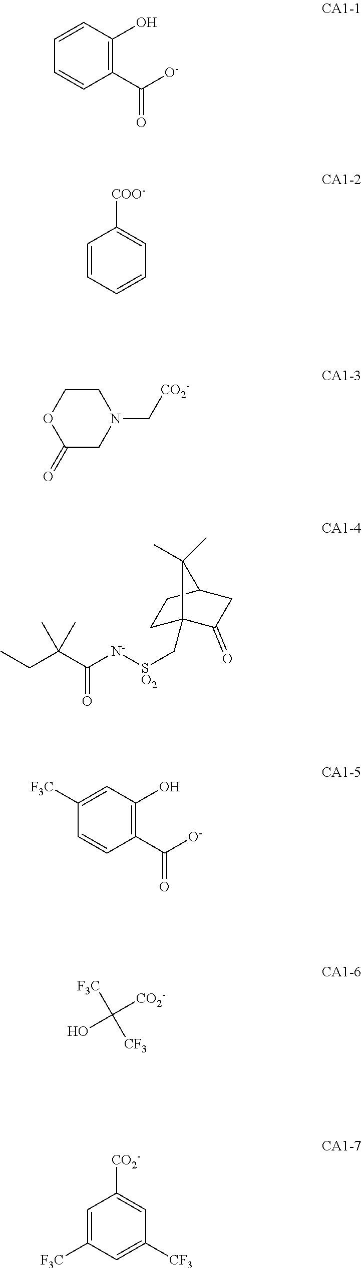

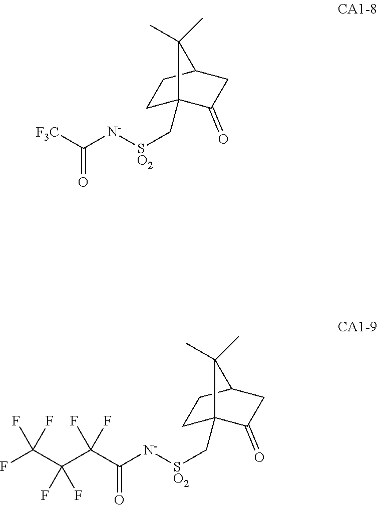 [SMILES: CC1(C)C2CCC1(CS(=O)(=O)[N-]C(=O)C(F)(F)F)C(=O)C2 ; CC1(C)C2CCC1(CS(=O)(=O)[N-]C(=O)C(F)(F)C(F)(F)C(F)(F)F)C(=O)C2]

### [Second Acid Diffusion Control Agent]

The structures of second cations CQ₂-1, CQ₂-2, and CQ₂-4 to CQ₂-9, a cation CQ₂-3 not corresponding to the second cation, and second anions CA2-1 to CA2-5 of the second acid diffusion control agent are shown below.

The combinations of the second cation and the second anion of the second acid diffusion control agent in each of the examples and the comparative examples are shown below in Tables 2 to 4.

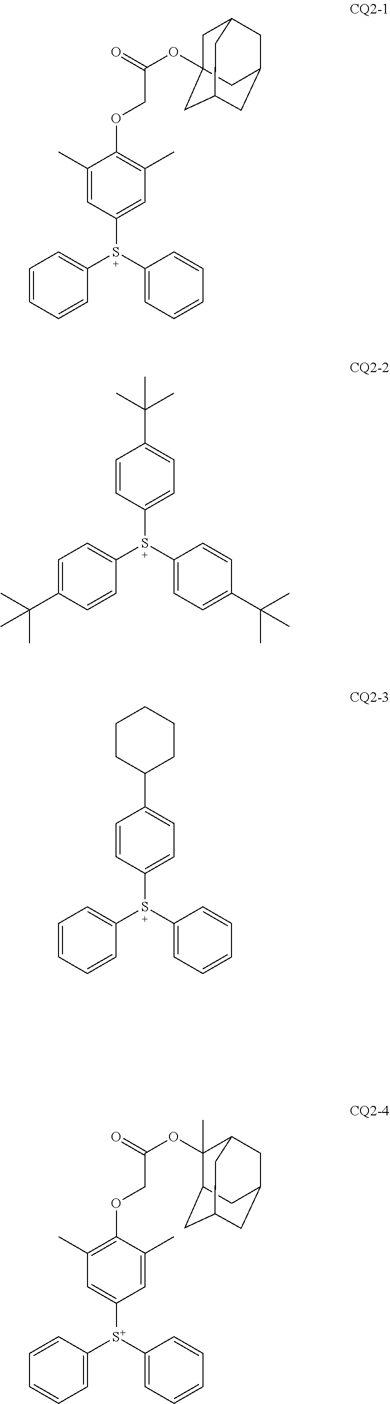

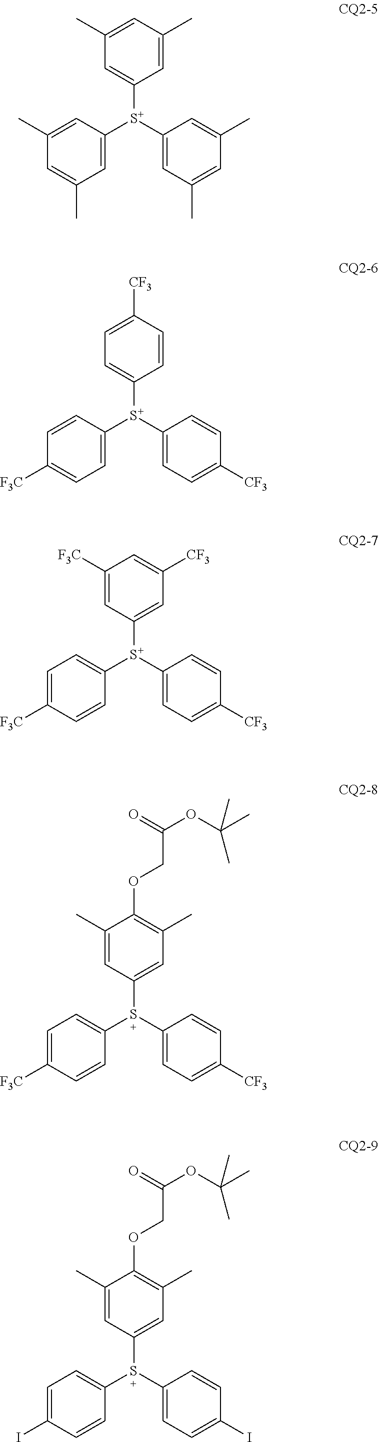

The structures of the second anions CA2-1 to CA2-5 of the second acid diffusion control agent are shown below.

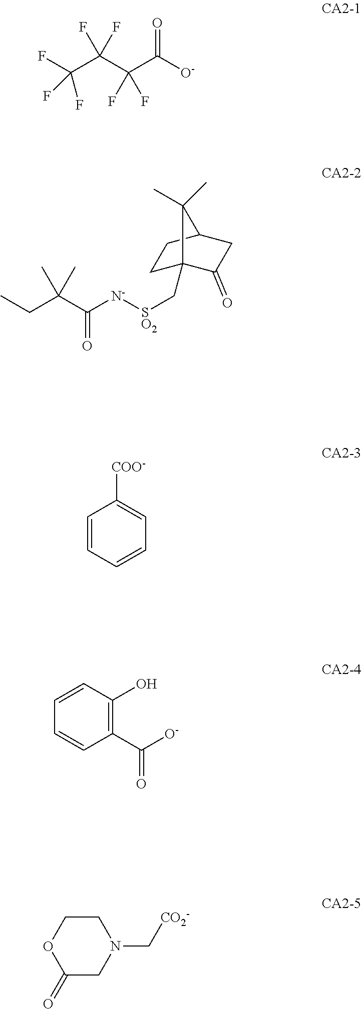

[Bound Product] The structures of anions CA3-1 to CA3-6 of bound products used in the examples are shown below.

In the following structural formulae, a numerical value of −1.50 or less represents an acid dissociation constant A, and a numerical value of more than −1.50 represents an acid dissociation constant B when the bound product is a bound product of the photoacid generator and the first acid diffusion control agent, and represents an acid dissociation constant C when the bound product is a bound product of the photoacid generator and the second acid diffusion control agent.

The cation in the bound product is selected from the first cations CQ₁-4 to CQ₁-12 or the second cations CQ₂-1, CQ₂-2, and CQ₂-4 to CQ₂-9, and combinations with the anions CA3-1 to CA3-6 are shown below in Tables 2 to 4.

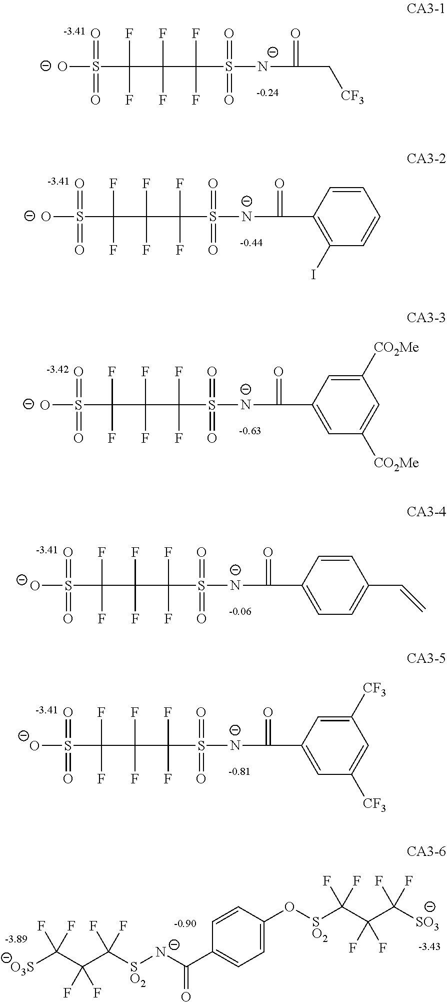

### [Solvent]

For the preparation of a resist solution, a solvent prepared by mixing the following solvents at the following ratio was used.
S1: propylene glycol monomethyl ether acetate
S2: propylene glycol monomethyl ether
S3: γ-butyrolactone
S1/S2/S3=85/10/5

These components were mixed at ratios shown in Tables 2 to 4 such that the total amount of components other than the solvent was 1.5% by mass based on the total mass of the resist composition. The resulting liquid mixture was then filtered through a polyethylene filter with a pore size of 0.03 μm to prepare a resist composition. The resist compositions were used in the examples and the comparative examples.

### [Pattern Formation and Evaluation]

### [Pattern Formation by EUV Exposure and Organic Solvent Development]

A composition AL412 for forming an underlayer film (manufactured by Brewer Science) was applied to a silicon wafer (12 inches) and was baked at 205° C. for 60 seconds to form an underlying film with a thickness of 5 nm. The resist composition prepared as described above was applied thereon and was baked at 90° C. for 60 seconds to form a resist film with a thickness of 35 nm.

The wafer coated with the resist film was subjected to pattern exposure using an EUV exposure apparatus (Micro Exposure Tool, NA (numerical aperture) 0.3, Quadrupole, outer sigma 0.68, inner sigma 0.36) manufactured by Exitech. An exposure mask with a line width of 20 nm having a 1:1 line-and-space pattern was used.

The exposed resist film was baked at 90° C. for 60 seconds, was then developed with n-butyl acetate for 30 seconds, and was spin-dried to form a negative-type pattern.

### [LWR Performance Evaluation]

The line-and-space pattern resolved at the optimum exposure level was observed from above the pattern using a critical dimension scanning electron microscope (SEM (Hitachi, Ltd., S-9380II)). The line width of the line-and-space pattern was measured at an arbitrary position, and 3σ (nm) was calculated wherein σ denotes the standard deviation of all the measured values. LWR was evaluated from the value of 3σ according to the following evaluation criteria.

LWR was evaluated based on the following five criteria. 3 or more indicates good LWR performance, 4 or more is preferred, and 5 is more preferred.

### (Evaluation Criteria)

“5”: 3σ of the variation is 3.0 nm or less.
“4”: 3σ of the variation is more than 3.0 nm and 3.5 nm or less.
“3”: 3σ of the variation is more than 3.5 nm and 4.0 nm or less.
“2”: 3σ of the variation is more than 4.0 nm and 4.5 nm or less.
“1”: 3σ of the variation is more than 4.5 nm.

### [Results]

Tables 2 to 4 show the evaluation results.

Examples 1 to 25 in Table 2 show the evaluation results of resist compositions each containing one photoacid generator, one first acid diffusion control agent, and one second acid diffusion control agent.

Examples 26 to 55 in Table 3 show the evaluation results of the examples using a bound product in which the anion in the photoacid generator and the second anion in the second acid diffusion control agent are bonded via a covalent bond. Bound products in Examples 26 to 55 are composed of an anion shown in the column “Structure (bound product)” and cation(s) shown in the second cation column in a number corresponding to the number of anion moieties in the anion. Examples 50 to 52 are embodiments including two bound products, and Examples 53 to 55 are embodiments including, in addition to the bound product, another photoacid generator that does not form a bound product.

Examples 56 to 59 in Table 4 show the evaluation results of the examples using a bound product in which the anion in the photoacid generator and the first anion in the first acid diffusion control agent are bonded via a covalent bond. Bound products in Examples 56 to 59 are composed of an anion shown in the column “Structure (bound product)” and cation(s) shown in the first cation column in a number corresponding to the number of anion moieties in the anion.

In the table, the numerical value in the column “wt %” of the column “Q₁ amount” of the column “First acid diffusion control agent (Q₁)” indicates the first acid diffusion control agent content (% by mass) based on the solid content of the resist composition.

In Table 2, the numerical value in the column “wt %” of the column “Q₂ amount” of the column “Second acid diffusion control agent (Q₂)” indicates the second acid diffusion control agent content (% by mass) based on the solid content of the resist composition.

In Table 2, the numerical value in the column “wt %” of the column “Photoacid generator” indicates the photoacid generator content (% by mass) based on the solid content of the resist composition.

In Tables 3 and 4, the numerical value in the column “wt %” of the column “Photoacid generator” indicates the bound product content (% by mass) based on the solid content of the resist composition.

In Tables 2 to 4, the numerical value in the column “wt %” of the column “Specific resin” indicates the specific resin content (% by mass) based on the solid content of the resist composition.

In the tables, the numerical value in the column “mol %” of the column “Q₁ amount” of the column “First acid diffusion control agent (Q₁)” indicates the first acid diffusion control agent content (% by mole) based on the total number of moles of the photoacid generator, the first acid diffusion control agent, and the second acid diffusion control agent.

In the tables, the numerical values in the column “Anion pKa(A)”, the column “Anion pKa(B)”, and the column “Anion pKa(C)” indicate the acid dissociation constant A, the acid dissociation constant B, and the acid dissociation constant C, respectively.

In the tables, the numerical value in the column “C log P value” of the column “First cation” and the column “Second cation” indicates the C log P value of the first cation and the C log P value of the second cation, respectively. The C log P value is calculated using ChemDrawProfessional (version 20.1.1.125, manufactured by PerkinElmer, Inc.).

TABLE 2

| | First acid diffusion control agent (Q1) | | | | | | Second acid diffusion control agent (Q2) | | | |
|---|---|---|---|---|---|---|---|---|---|---|
| | | | First anion | | | | | | Second anion | |
| | First cation | | | Anion | Amount of Q1 | | Second cation | | Anion | |
| | Structure | ClogP | Structure | pKa(B) | wt % | mol % | Structure | ClogP | pKa(C) | Structure |
| 1 | CQ1-1 | 8.571 | CA1-1 | 3.01 | 3.00 | 22 | CQ2-1 | 8.571 | 0.75 | CA2-1 |
| 2 | CQ1-2 | 5.377 | CA1-1 | 3.01 | 1.80 | 10 | CQ2-2 | 10.860 | 4.19 | CA2-2 |
| 3 | CQ1-3 | 6.260 | CA1-1 | 3.01 | 2.11 | 10 | CQ2-2 | 10.860 | 4.19 | CA2-2 |
| 4 | CQ1-2 | 5.377 | CA1-1 | 3.01 | 1.80 | 10 | CQ2-3 | 7.997 | 4.19 | CA2-2 |
| 5 | CQ1-4 | 5.806 | CA1-1 | 3.01 | 2.04 | 10 | CQ2-3 | 7.997 | 4.19 | CA2-2 |
| 1 | CQ1-4 | 5.806 | CA1-1 | 3.01 | 2.04 | 10 | CQ2-2 | 10.860 | 4.19 | CA2-2 |
| 2 | CQ1-4 | 5.806 | CA1-2 | 4.20 | 1.97 | 10 | CQ2-2 | 10.860 | 4.20 | CA2-3 |
| 3 | CQ1-4 | 5.806 | CA1-3 | 2.01 | 2.14 | 10 | CQ2-2 | 10.860 | 2.45 | CA2-4 |
| 4 | CQ1-4 | 5.806 | CA1-4 | 4.19 | 2.91 | 10 | CQ2-2 | 10.860 | 2.01 | CA2-5 |
| 5 | CQ1-4 | 5.806 | CA1-1 | 3.01 | 2.04 | 10 | CQ2-2 | 10.860 | 4.20 | CA2-3 |
| 6 | CQ1-5 | 5.520 | CA1-2 | 4.20 | 2.12 | 10 | CQ2-2 | 10.860 | 2.45 | CA2-4 |
| 7 | CQ1-6 | 5.466 | CA1-3 | 2.01 | 1.95 | 10 | CQ2-2 | 10.860 | 2.01 | CA2-5 |
| 8 | CQ1-7 | 5.876 | CA1-1 | 3.01 | 1.86 | 10 | CQ2-4 | 9.090 | 4.19 | CA2-2 |
| 9 | CQ1-8 | 5.875 | CA1-2 | 4.20 | 2.72 | 10 | CQ2-5 | 8.371 | 4.19 | CA2-2 |
| 10 | CQ1-4 | 5.806 | CA1-1 | 3.01 | 2.04 | 10 | CQ2-6 | 8.026 | 4.19 | CA2-2 |
| 11 | CQ1-4 | 5.806 | CA1-2 | 4.20 | 1.97 | 10 | CQ2-7 | 8.909 | 4.19 | CA2-2 |
| 12 | CQ1-5 | 5.520 | CA1-2 | 4.20 | 2.12 | 10 | CQ2-8 | 8.915 | 4.19 | CA2-2 |
| 13 | CQ1-8 | 5.875 | CA1-2 | 4.20 | 2.72 | 10 | CQ2-9 | 9.395 | 4.19 | CA2-2 |
| 14 | CQ1-4 | 5.806 | CA1-5 | 2.45 | 2.35 | 10 | CQ2-2 | 10.86 | 4.19 | CA2-2 |
| 15 | CQ1-5 | 5.520 | CA1-5 | 2.45 | 2.19 | 10 | CQ2-7 | 8.909 | 4.19 | CA2-2 |
| 16 | CQ1-5 | 5.520 | CA1-5 | 2.45 | 3.50 | 16 | CQ2-7 | 8.909 | 4.19 | CA2-2 |
| 17 | CQ1-5 | 5.520 | CA1-5 | 2.45 | 3.28 | 15 | CQ2-7 | 8.909 | 4.19 | CA2-2 |
| 18 | CQ1-5 | 5.520 | CA1-6 | 0.84 | 3.32 | 10 | CQ2-6 | 8.026 | 4.19 | CA2-2 |
| 19 | CQ1-6 | 5.466 | CA1-7 | 3.34 | 3.59 | 10 | CQ2-7 | 8.909 | 4.19 | CA2-2 |
| 20 | CQ1-7 | 5.876 | CA1-8 | −0.97 | 4.08 | 10 | CQ2-8 | 8.915 | 4.19 | CA2-2 |
| 21 | CQ1-8 | 5.875 | CA1-9 | 0.15 | 5.14 | 10 | CQ2-9 | 9.395 | 4.19 | CA2-2 |
| 22 | CQ1-9 | 5.683 | CA1-5 | 2.45 | 3.83 | 10 | CQ2-7 | 8.909 | 4.19 | CA2-2 |
| 23 | CQ1-10 | 5.526 | CA1-9 | 0.15 | 4.47 | 10 | CQ2-9 | 9.395 | 4.19 | CA2-2 |
| 24 | CQ1-11 | 5.756 | CA1-7 | 3.34 | 3.88 | 10 | CQ2-6 | 8.026 | 4.19 | CA2-2 |
| 25 | CQ1-12 | 5.786 | CA1-8 | −0.97 | 5.36 | 10 | CQ2-8 | 8.915 | 4.19 | CA2-2 |

| | Second acid diffusion control agent (Q2) | Photoacid generator | | | | | |
|---|---|---|---|---|---|---|---|
| | Amount of Q2 | | Anion | | Specific resin | | Effect |
| | wt % | Structure | pKa(A) | wt % | Structure | wt % | LWR |
| 1 | 8.00 | P-1 | −2.78 | 8.00 | E-1 | 81.0 | 1 |
| 2 | 11.80 | P-2 | −2.06 | 15.94 | E-2 | 70.5 | 2 |
| 3 | 11.80 | P-2 | −2.06 | 15.94 | E-2 | 70.2 | 2 |
| 4 | 10.46 | P-2 | −2.06 | 15.94 | E-2 | 71.8 | 1 |
| 5 | 10.46 | P-2 | −2.06 | 15.94 | E-2 | 71.6 | 2 |
| 1 | 11.80 | P-2 | −2.06 | 15.90 | E-2 | 70.3 | 3 |
| 2 | 8.12 | P-3 | −2.59 | 19.30 | E-12 | 70.6 | 3 |
| 3 | 8.83 | P-4 | −3.57 | 15.40 | E-13 | 73.6 | 3 |
| 4 | 9.16 | P-5 | −1.99 | 22.10 | E-14 | 65.8 | 3 |
| 5 | 8.12 | P-2 | −2.06 | 19.30 | E-2 | 70.5 | 3 |
| 6 | 8.83 | P-2 | −2.06 | 15.40 | E-3 | 73.7 | 3 |
| 7 | 9.16 | P-2 | −2.06 | 22.10 | E-4 | 66.8 | 3 |
| 8 | 13.07 | P-2 | −2.06 | 15.90 | E-3 | 69.2 | 3 |
| 9 | 10.50 | P-2 | −2.06 | 15.90 | E-4 | 70.9 | 3 |
| 10 | 12.35 | P-2 | −2.06 | 15.90 | E-2 | 69.7 | 4 |
| 11 | 13.41 | P-2 | −2.06 | 15.90 | E-5 | 68.7 | 4 |
| 12 | 13.75 | P-2 | −2.06 | 15.90 | E-8 | 68.2 | 4 |
| 13 | 15.55 | P-2 | −2.06 | 15.90 | E-9 | 65.8 | 4 |
| 14 | 11.80 | P-2 | −2.06 | 15.90 | E-2 | 70.0 | 4 |
| 15 | 13.41 | P-2 | −2.06 | 15.90 | E-2 | 68.5 | 5 |
| 16 | 11.07 | P-2 | −2.06 | 15.90 | E-2 | 69.5 | 4 |
| 17 | 11.47 | P-2 | −2.06 | 15.90 | E-2 | 69.4 | 5 |
| 18 | 12.35 | P-2 | −2.06 | 15.90 | E-3 | 68.4 | 5 |
| 19 | 13.41 | P-2 | −2.06 | 15.90 | E-5 | 67.1 | 5 |
| 20 | 13.75 | P-2 | −2.06 | 15.90 | E-6 | 66.3 | 5 |
| 21 | 15.55 | P-2 | −2.06 | 15.90 | E-8 | 63.4 | 5 |
| 22 | 13.41 | P-2 | −2.06 | 15.90 | E-9 | 66.9 | 5 |
| 23 | 15.55 | P-2 | −2.06 | 15.90 | E-10 | 64.1 | 5 |
| 24 | 12.35 | P-2 | −2.06 | 15.90 | E-11 | 67.9 | 5 |
| 25 | 13.75 | P-2 | −2.06 | 15.90 | E-2 | 65.0 | 4 |

TABLE 3

| | | | | | | | Second acid diffusion | | | | | | | | |
|---|---|---|---|---|---|---|---|---|---|---|---|---|---|---|---|
| | First acid diffusion | | | | | | control agent (Q2) | | | Photoacid generator | | | | | |
| | control agent (Q1) | | | | | | | | Second | Struc- | | | | | |
| | First cation | | First anion | | Amount | | Second cation | | anion | ture | | | Specific resin | | |
| | Struc- | | Struc- | Anion | of Q1 | | Struc- | | Anion | (bound | Anion | | Struc- | | Effect |
| | ture | ClogP | ture | pKa(B) | wt % | mol % | ture | ClogP | pKa(C) | product) | pKa(A) | wt % | ture | wt % | LWR |
| 26 | CQ1-4 | 5.806 | CA1-4 | 4.19 | 2.91 | 10 | CQ2-2 | 10.860 | −0.24 | CA3-1 | −3.41 | 25.97 | E-2 | 71.1 | 3 |
| 27 | CQ1-5 | 5.520 | CA1-2 | 4.20 | 2.12 | 10 | CQ2-2 | 10.860 | −0.44 | CA3-2 | −3.41 | 28.40 | E-3 | 69.5 | 3 |
| 28 | CQ1-6 | 5.466 | CA1-3 | 2.01 | 1.95 | 10 | CQ2-2 | 10.860 | −0.63 | CA3-3 | −3.42 | 28.20 | E-5 | 69.9 | 3 |
| 29 | CQ1-7 | 5.876 | CA1-1 | 3.01 | 1.86 | 10 | CQ2-2 | 10.860 | 0.06 | CA3-4 | −3.41 | 26.40 | E-6 | 71.7 | 3 |
| 30 | CQ1-8 | 5.875 | CA1-2 | 4.20 | 2.72 | 10 | CQ2-4 | 9.090 | −0.81 | CA3-5 | −3.41 | 31.91 | E-2 | 65.4 | 3 |
| 31 | CQ1-9 | 5.683 | CA1-1 | 3.01 | 2.25 | 10 | CQ2-5 | 8.371 | −0.63 | CA3-3 | −3.42 | 24.79 | E-2 | 73.0 | 3 |
| 32 | CQ1-4 | 5.806 | CA1-2 | 4.20 | 1.97 | 10 | CQ2-6 | 8.026 | 0.06 | CA3-4 | −3.41 | 27.82 | E-2 | 70.2 | 4 |
| 33 | CQ1-5 | 5.520 | CA1-1 | 3.01 | 2.25 | 10 | CQ2-7 | 8.909 | −0.44 | CA3-2 | −3.41 | 32.60 | E-8 | 65.2 | 4 |
| 34 | CQ1-6 | 5.466 | CA1-4 | 4.19 | 2.71 | 10 | CQ2-8 | 8.915 | −0.24 | CA3-1 | −3.41 | 31.10 | E-10 | 66.2 | 4 |
| 35 | CQ1-7 | 5.876 | CA1-3 | 2.01 | 1.96 | 10 | CQ2-9 | 9.395 | −0.24 | CA3-1 | −3.41 | 35.74 | E-11 | 62.3 | 4 |
| 36 | CQ1-4 | 5.806 | CA1-8 | −0.97 | 2.90 | 10 | CQ2-2 | 10.86 | 0.06 | CA3-4 | −3.41 | 26.36 | E-2 | 70.7 | 4 |
| 37 | CQ1-6 | 5.466 | CA1-9 | 0.15 | 3.16 | 10 | CQ2-2 | 10.86 | −0.24 | CA3-1 | −3.41 | 26.00 | E-5 | 70.8 | 4 |
| 38 | CQ1-5 | 5.520 | CA1-5 | 2.45 | 2.19 | 10 | CQ2-6 | 8.026 | −0.24 | CA3-1 | −3.41 | 27.42 | E-2 | 70.4 | 5 |
| 39 | CQ1-5 | 5.520 | CA1-5 | 2.45 | 3.50 | 16 | CQ2-6 | 8.026 | −0.24 | CA3-1 | −3.41 | 25.59 | E-2 | 70.9 | 4 |
| 40 | CQ1-5 | 5.520 | CA1-5 | 2.45 | 3.28 | 15 | CQ2-6 | 8.026 | −0.24 | CA3-1 | −3.41 | 25.89 | E-2 | 70.8 | 5 |
| 41 | CQ1-5 | 5.520 | CA1-6 | 0.84 | 3.32 | 10 | CQ2-7 | 8.909 | −0.44 | CA3-2 | −3.41 | 32.58 | E-3 | 64.1 | 5 |
| 42 | CQ1-6 | 5.466 | CA1-7 | 3.34 | 3.59 | 10 | CQ2-9 | 9.395 | −0.81 | CA3-5 | −3.41 | 38.39 | E-4 | 58.0 | 5 |
| 43 | CQ1-7 | 5.876 | CA1-8 | −0.97 | 4.08 | 10 | CQ2-8 | 8.915 | −0.24 | CA3-1 | −3.41 | 31.10 | E-5 | 64.8 | 5 |
| 44 | CQ1-8 | 5.875 | CA1-9 | 0.15 | 5.14 | 10 | CQ2-8 | 8.915 | −0.44 | CA3-2 | −3.41 | 33.50 | E-6 | 61.4 | 5 |
| 45 | CQ1-9 | 5.683 | CA1-5 | 2.45 | 3.83 | 10 | CQ2-7 | 8.909 | −0.81 | CA3-5 | −3.41 | 32.80 | E-7 | 63.4 | 5 |
| 46 | CQ1-10 | 5.526 | CA1-9 | 0.15 | 4.47 | 10 | CQ2-6 | 8.026 | −0.44 | CA3-2 | −3.41 | 29.84 | E-10 | 65.7 | 5 |
| 47 | CQ1-11 | 5.756 | CA1-7 | 3.34 | 3.88 | 10 | CQ2-9 | 9.395 | −0.24 | CA3-1 | −3.41 | 35.74 | E-11 | 60.4 | 5 |
| 48 | CQ1-12 | 5.786 | CA1-8 | −0.97 | 5.36 | 10 | CQ2-7 | 8.909 | −0.24 | CA3-1 | −3.41 | 30.17 | E-2 | 64.5 | 4 |
| 49 | CQ1-8 | 5.875 | CA1-5 | 2.45 | 3.35 | 10 | CQ2-6 | 8.026 | −0.90 | CA3-6 | −3.89, −3.43 | 28.68 | E-2 | 68.0 | 5 |
| 50 | CQ1-5 | 5.520 | CA1-5 | 2.45 | 2.18 | 10 | CQ2-8 | 8.915 | −0.44 | CA3-2 | −3.41 | 10.05 | E-2 | 65.1 | 5 |
| | | | | | | | | | −0.90 | CA3-6 | −3.89, −3.43 | 22.63 | | | |
| 51 | CQ1-5 | 5.520 | CA1-6 | 0.84 | 2.21 | 10 | CQ2-8 | 8.915 | −0.44 | CA3-2 | −3.41 | 10.05 | E-2 | 65.1 | 5 |
| | | | | | | | | | −0.90 | CA3-6 | −3.89, −3.43 | 22.63 | | | |
| 52 | CQ1-5 | 5.520 | CA1-7 | 3.34 | 2.31 | 10 | CQ2-8 | 8.915 | −0.44 | CA3-2 | −3.41 | 10.05 | E-2 | 65.0 | 5 |
| | | | | | | | | | −0.90 | CA3-6 | −3.89, −3.43 | 22.63 | | | |
| 53 | CQ1-5 | 5.520 | CA1-7 | 3.34 | 2.31 | 10 | CQ2-8 | 8.915 | −0.44 | CA3-2 | −3.41 | 25.7 | E-2 | 62.1 | 5 |
| | | | | | | | | | — | P-8 | −1.99 | 9.87 | | | |
| 54 | CQ1-5 | 5.520 | CA1-7 | 3.34 | 2.31 | 10 | CQ2-8 | 8.915 | −0.44 | CA3-2 | −3.41 | 25.7 | E-2 | 64.5 | 5 |
| | | | | | | | | | — | P-6 | −3.29 | 7.47 | | | |
| 55 | CQ1-5 | 5.520 | CA1-7 | 3.34 | 2.31 | 10 | CQ2-8 | 8.915 | −0.44 | CA3-2 | −3.41 | 25.7 | E-2 | 64.5 | 5 |
| | | | | | | | | | — | P-7 | −2.70 | 7.47 | | | |

TABLE 4

| | Second acid diffusion | | | | | | First acid diffusion | | | | | | | | |
|---|---|---|---|---|---|---|---|---|---|---|---|---|---|---|---|
| | control agent (Q2) | | | | | | control agent (Q1) | | | Photoacid generator | | | | | |
| | | | | | | | | | First | Struc- | | | | | |
| | Second cation | | First anion | | Amount | | First cation | | anion | ture | | | Specific resin | | |
| | Struc- | | Struc- | Anion | of Q2 | | Struc- | | Anion | (bound | Anion | | Struc- | | Effect |
| | ture | ClogP | ture | pKa(C) | wt % | mol % | ture | ClogP | pKa(B) | product) | pKa(A) | wt % | ture | wt % | LWR |
| 56 | CQ2-2 | 10.860 | CA2-2 | 4.19 | 3.42 | 10 | CQ1-4 | 5.806 | −0.24 | CA3-1 | −3.41 | 21.34 | E-2 | 75.2 | 3 |
| 57 | CQ2-4 | 9.090 | CA2-2 | 4.19 | 3.79 | 10 | CQ1-4 | 5.806 | −0.24 | CA3-1 | −3.41 | 21.34 | E-2 | 74.9 | 3 |
| 58 | CQ2-6 | 8.026 | CA2-2 | 4.19 | 3.58 | 10 | CQ1-4 | 5.806 | −0.24 | CA3-1 | −3.41 | 21.34 | E-2 | 75.1 | 4 |
| 59 | CQ2-6 | 8.026 | CA2-4 | 2.45 | 2.72 | 10 | CQ1-4 | 5.806 | −0.24 | CA3-1 | −3.41 | 21.34 | E-2 | 75.9 | 4 |

The results in Tables 2 to 4 showed that the resist compositions according to the examples of the present invention can form a pattern with a low LWR.

On the other hand, the resist compositions of the comparative examples in which the C log P value of at least one of the first cation or the second cation was outside the range were not satisfactory in terms of the LWR of the pattern.

A comparison of Examples 15 to 17 and the like showed that the present invention has higher advantages when the first acid diffusion control agent content is 15% by mole or less based on the total number of moles of the photoacid generator, the first acid diffusion control agent, and the second acid diffusion control agent.

A comparison of Examples 1 to 9 and 10 to 13 and the like showed that the present invention has higher advantages when the second cation is a cation containing at least one selected from the group consisting of a fluorine atom and an iodine atom.

A comparison of Examples 10 to 13 and 17 to 24 and the like showed that the present invention has higher advantages when the first anion is an anion containing at least one selected from the group consisting of a fluorine atom and an iodine atom.

A comparison of Examples 24 and 25 and the like showed that the present invention has higher advantages when both of the first cation and the second cation were sulfonium cations.

When the LWR performance was evaluated using a pattern formed by alkaline aqueous solution development as described in the following [Pattern Formation by EUV Exposure and Alkaline Aqueous Solution Development] instead of the pattern forming method described in the [Pattern Formation by EUV Exposure and Organic Solvent Development], it was confirmed that the same effects as in the examples described in Tables 2 to 4 were obtained.

### [Pattern Formation by EUV Exposure and Alkaline Aqueous Solution Development]

A composition AL412 for forming an underlayer film (manufactured by Brewer Science) was applied to a silicon wafer (12 inches) and was baked at 205° C. for 60 seconds to form an underlying film with a thickness of 20 nm. The resist composition prepared as described above was applied thereon and was baked at 100° C. for 60 seconds to form a resist film with a thickness of 30 nm.

The wafer coated with the resist film was subjected to pattern exposure using an EUV exposure apparatus (Micro Exposure Tool, NA (numerical aperture) 0.3, Quadrupole, outer sigma 0.68, inner sigma 0.36) manufactured by Exitech. An exposure mask with a line width of 20 nm having a 1:1 line-and-space pattern was used.

The exposed resist film was baked at 90° C. for 60 seconds, was then developed with aqueous tetramethylammonium hydroxide (2.38% by mass) for 30 seconds, and was then rinsed with pure water for 30 seconds. This was then spin-dried to form a positive-type pattern.

What is claimed is:

1. An actinic ray-sensitive or radiation-sensitive resin composition comprising:
a resin whose polarity is increased by the action of an acid;
one or more photoacid generators;
a first acid diffusion control agent composed of a first cation and a first anion; and
a second acid diffusion control agent composed of a second cation and a second anion,
wherein the photoacid generator is composed of a cation and an anion and generates an acid upon irradiation with an actinic ray or radiation,
an acidic compound produced by replacing the cation in the photoacid generator with a proton has an acid dissociation constant A of −1.50 or less,
an acidic compound produced by replacing the first cation of the first acid diffusion control agent with a proton has an acid dissociation constant B of more than −1.50, and an acidic compound produced by replacing the second cation of the second acid diffusion control agent with a proton has an acid dissociation constant C of more than −1.50,
the first cation has a C log P value in the range of 5.400 to 6.000,
the second cation has a C log P value of 8.000 or more, and
the one or more photoacid generators may be bonded to the first acid diffusion control agent or the second acid diffusion control agent via a covalent bond.

2. The actinic ray-sensitive or radiation-sensitive resin composition according to claim 1, wherein a first acid diffusion control agent content is 15% by mole or less based on a total number of moles of the photoacid generator, the first acid diffusion control agent, and the second acid diffusion control agent.

3. The actinic ray-sensitive or radiation-sensitive resin composition according to claim 1, wherein the second cation is a cation containing at least one selected from the group consisting of a fluorine atom and an iodine atom.

4. The actinic ray-sensitive or radiation-sensitive resin composition according to claim 1, wherein the first anion is an anion containing at least one selected from the group consisting of a fluorine atom and an iodine atom.

5. The actinic ray-sensitive or radiation-sensitive resin composition according to claim 1, wherein both the first cation and the second cation are sulfonium cations.

6. A resist film formed by using the actinic ray-sensitive or radiation-sensitive resin composition according to claim 1.

7. A pattern forming method comprising:
a step 1 of forming a resist film on a substrate using the actinic ray-sensitive or radiation-sensitive resin composition according to claim 1;
a step 2 of exposing the resist film; and
a step 3 of developing the exposed resist film using a developer to form a resist pattern.

8. A method for producing an electronic device, comprising the pattern forming method according to claim 7.

9. The actinic ray-sensitive or radiation-sensitive resin composition according to claim 2, wherein the second cation is a cation containing at least one selected from the group consisting of a fluorine atom and an iodine atom.

10. The actinic ray-sensitive or radiation-sensitive resin composition according to claim 2, wherein the first anion is an anion containing at least one selected from the group consisting of a fluorine atom and an iodine atom.

11. The actinic ray-sensitive or radiation-sensitive resin composition according to claim 2, wherein both the first cation and the second cation are sulfonium cations.

12. A resist film formed by using the actinic ray-sensitive or radiation-sensitive resin composition according to claim 2.

13. A pattern forming method comprising:
a step 1 of forming a resist film on a substrate using the actinic ray-sensitive or radiation-sensitive resin composition according to claim 2;
a step 2 of exposing the resist film; and
a step 3 of developing the exposed resist film using a developer to form a resist pattern.

14. A method for producing an electronic device, comprising the pattern forming method according to claim 13.

15. The actinic ray-sensitive or radiation-sensitive resin composition according to claim 3, wherein the first anion is an anion containing at least one selected from the group consisting of a fluorine atom and an iodine atom.

16. The actinic ray-sensitive or radiation-sensitive resin composition according to claim 3, wherein both the first cation and the second cation are sulfonium cations.

17. A resist film formed by using the actinic ray-sensitive or radiation-sensitive resin composition according to claim 3.

18. A pattern forming method comprising:
a step 1 of forming a resist film on a substrate using the actinic ray-sensitive or radiation-sensitive resin composition according to claim 3;
a step 2 of exposing the resist film; and
a step 3 of developing the exposed resist film using a developer to form a resist pattern.

19. A method for producing an electronic device, comprising the pattern forming method according to claim 3.

20. The actinic ray-sensitive or radiation-sensitive resin composition according to claim 4, wherein both the first cation and the second cation are sulfonium cations.
